(19) United States
(12) Patent Application Publication
(10) Pub. No.: US 2022/0087505 A1
(43) Pub. Date: Mar. 24, 2022

(54) ENDOSCOPE APPARATUS AND BENDING MEMBER FOR ENDOSCOPE

(71) Applicant: OLYMPUS CORPORATION, Tokyo (JP)

(72) Inventor: Yasuo HIRATA, Tokyo (JP)

(73) Assignee: OLYMPUS CORPORATION, Tokyo (JP)

(21) Appl. No.: 17/540,287

(22) Filed: Dec. 2, 2021

Related U.S. Application Data
(63) Continuation of application No. PCT/JP2020/019427, filed on May 15, 2020.

(51) Int. Cl. A61B 1/005; A61B 1/00

(52) U.S. Cl. CPC A61B 1/0057; A61B 1/00135; A61B 1/0011

(57) ABSTRACT

An endoscope apparatus includes a bending portion bent by at least one bending wire, the bending portion including a first region and a second region in a longitudinal direction of the bending portion, the second region being a region provided on a proximal end side of the first region and configured to be bent following bending of the first region, the at least one bending wire being held on a proximal end side of the second region, the first region and the second region being integrally formed.

### CROSS REFERENCE TO RELATED APPLICATION

This application is a continuation application of PCT/JP2020/019427 filed on May 15, 2020, the entire contents of which are incorporated herein by this reference.

### BACKGROUND OF THE INVENTION

### 1. Field of the Invention

The present invention relates to an endoscope apparatus and a bending member for endoscope.

### 2. Description of the Related Art

Endoscopes are widely used in industrial and medical fields. The endoscope has an active bending portion at an insertion portion, and a user who is an examiner can bend the active bending portion. The user can insert the insertion portion into an object and observe an inside in a desired direction by bending the active bending portion. The active bending portion includes a plurality of bending pieces in order to be able to bend in a desired direction according to the operation of the user. A plurality of the bending pieces are configured to be fixed so that, for example, two adjacent bending pieces can be rotated around a predetermined axis by two rivets.

When the insertion portion is inserted into the object while the active bending portion is bent, the active bending portion may be damaged. For example, in a case of an industrial endoscope, when a side surface of the active bending portion comes into contact with a tube wall in the object, the active bending portion that can bend in the up-down direction receives a reaction force in the left-right direction, and a load is applied to a plurality of the bending pieces in the active bending portion, so that the active bending portion may be damaged.

Therefore, Japanese Patent Application Laid-Open Publication No. 2015-119839 proposes an endoscope having a structure in which a tubular bending portion is provided on the proximal end side of the active bending portion of the insertion portion to reduce the load applied to the active bending portion.

### SUMMARY OF THE INVENTION

An endoscope apparatus of one aspect of the present invention is an endoscope apparatus, including a bending portion bent by at least one bending wire. The bending portion includes a first region and a second region in a longitudinal direction of the bending portion. The second region is a region provided on a proximal end side of the first region and configured to be bent following bending of the first region. The at least one bending wire is held on a proximal end side of the second region. The first region and the second region are integrally formed.

A bending member for endoscope according to one aspect of the present invention includes a first region and a second region in a longitudinal direction of a bending portion of an endoscope. The second region is a region provided on a proximal end side of the first region and configured to be bent following bending of the first region. The first region and the second region are integrally formed.

### BRIEF DESCRIPTION OF THE DRAWINGS

FIG. 1 is a configuration diagram of an endoscope system according to a first embodiment;

FIG. 2 is a configuration diagram of a bending portion of an insertion portion according to the first embodiment;

FIG. 3 is a cross-sectional view of a pipe distal end connecting portion when the pipe is viewed from the distal end side, according to the first embodiment;

FIG. 4 is a cross-sectional view of a portion of the pipe where two wire receivers are formed when the pipe is viewed from the distal end side, according to the first embodiment;

FIG. 5 is a cross-sectional view of a portion where a lower side slit of the pipe is formed when the pipe is viewed from the distal end side, according to the first embodiment;

FIG. 6 is a cross-sectional view of a portion where an upper side slit of the pipe is formed when the pipe is viewed from the distal end side, according to the first embodiment;

FIG. 7 is a cross-sectional view of a portion where a left side slit of the pipe is formed when the pipe is viewed from the distal end side, according to the first embodiment;

FIG. 8 is a cross-sectional view of a portion where a right side slit of the pipe is formed when the pipe is viewed from the distal end side, according to the first embodiment;

FIG. 9 is a diagram for explaining an action when the distal end portion of the insertion portion of the endoscope having a structure of a comparative example is pushed into an L-shaped pipe;

FIG. 10 is a diagram for explaining an action when the insertion portion of the endoscope having the structure of the comparative example is pulled out from the L-shaped pipe;

FIG. 11 is a diagram for explaining an action when the distal end portion of the insertion portion of the endoscope having a structure of the first embodiment is pushed into the L-shaped pipe;

FIG. 12 is a diagram for explaining an action when the distal end portion of the insertion portion of the endoscope having the structure of the first embodiment is pulled out from the L-shaped pipe;

FIG. 13 is a configuration diagram of a bending portion and a flexible tube portion of an insertion portion according to a modification 1 of the first embodiment;

FIG. 14 is a configuration diagram of a bending portion of an insertion portion according to a modification 2 of the first embodiment;

FIG. 15 is a configuration diagram of a bending portion of an insertion portion according to a modification 3 of the first embodiment;

FIG. 16 is a configuration diagram of a bending portion of an insertion portion according to a modification 4 of the first embodiment;

FIG. 17 is a configuration diagram of a bending portion of an insertion portion according to a modification 5 of the first embodiment;

FIG. 18 is a configuration diagram of a bending portion of an insertion portion according to a modification 6 of the first embodiment;

FIG. 19 is a configuration diagram of a bending portion of an insertion portion according to a modification 7 of the first embodiment;

FIG. 20 is a configuration diagram of a bending portion of an insertion portion according to a modification 8 of the first embodiment;

FIG. 21 is a configuration diagram of a bending portion of an insertion portion according to a modification 9 of the first embodiment;

FIG. 22 is a cross-sectional view of a pipe at a bending portion of an insertion portion according to a modification 9 of the first embodiment;

FIG. 23 is a cross-sectional view of a pipe before a slit or the like is formed according to a modification 9 of the first embodiment;

FIG. 24 is a configuration diagram of a bending portion of an insertion portion according to a modification 10 of the first embodiment;

FIG. 25 is a configuration diagram of a bending portion of an insertion portion according to a modification 11 of the first embodiment;

FIG. 26 is a cross-sectional view of a bending portion of an insertion portion according to the modification 11 of the first embodiment;

FIG. 27 is a configuration diagram of a bending portion of an insertion portion according to a modification 12 of the first embodiment;

FIG. 28 is a diagram for explaining an action when the distal end portion of the insertion portion of the endoscope having a structure of the modification 12 of the first embodiment is pushed into the L-shaped pipe;

FIG. 29 is a diagram for explaining an action when the distal end portion of the insertion portion of the endoscope having the structure of the modification 12 of the first embodiment is pulled out from the L-shaped pipe;

FIG. 30 is a perspective view of a coil used for a bending portion according to the second embodiment;

FIG. 31 is an assembly diagram of a bending portion according to a second embodiment;

FIG. 32 is a perspective view of a multi-lumen tube used in a bending portion according to a third embodiment;

FIG. 33 is a cross-sectional view of a portion where a lower side slit of the multi-lumen tube is formed when the multi-lumen tube is viewed from the distal end side, according to the third embodiment;

FIG. 34 is a cross-sectional view of a portion where an upper side slit of the multi-lumen tube is formed when the multi-lumen tube is viewed from the distal end side, according to the third embodiment;

FIG. 35 is a cross-sectional view of a portion where a left side slit of the multi-lumen tube is formed when the multi-lumen tube is viewed from the distal end side, according to the third embodiment;

FIG. 36 is a cross-sectional view of a portion where a right side slit of the multi-lumen tube is formed when the multi-lumen tube is viewed from the distal end side, according to the third embodiment;

FIG. 37 is a configuration diagram of a bending portion of an insertion portion according to a fourth embodiment;

FIG. 38 is a diagram for explaining an action when the distal end portion of the insertion portion of the endoscope having a structure of the fourth embodiment is pushed into the L-shaped pipe;

FIG. 39 is a diagram for explaining an action when the distal end portion of the insertion portion of the endoscope having a structure of the fourth embodiment is pushed into the L-shaped pipe;

FIG. 40 is a diagram for explaining an action when the distal end portion of the insertion portion of the endoscope having a structure of the fourth embodiment is pushed into the L-shaped pipe;

FIG. 41 is a configuration diagram of a bending portion of an insertion portion according to a modification 4-1 of the fourth embodiment;

FIG. 42 is a configuration diagram of a bending portion of an insertion portion according to a modification 4-2 of the fourth embodiment;

FIG. 43 is a configuration diagram showing a configuration of a bending portion of an insertion portion according to a modification 4-3 of the fourth embodiment;

FIG. 44 is a diagram for explaining fitting of two adjacent pipes according to the modification 4-3 of the fourth embodiment;

FIG. 45 is a configuration diagram of a bending portion of an insertion portion according to a first disclosure example;

FIG. 46 is an assembly diagram of a bending portion of a small-diameter insertion portion according to a second disclosure example;

FIG. 47 is a cross-sectional view of a bending portion in the longitudinal direction of the insertion portion according to the second disclosure example;

FIG. 48 is a cross-sectional view of a bending portion orthogonal to the longitudinal direction of the insertion portion according to the second disclosure example;

FIG. 49 is a cross-sectional view of a bending portion orthogonal to the longitudinal direction of the insertion portion according to the second disclosure example;

FIG. 50 is an assembly diagram showing a configuration of a bending portion of an insertion portion using a coil coarsely wound on the distal end side and tightly wound on the proximal end side, according to the second disclosure example;

FIG. 51 is an assembly diagram showing a configuration of a bending portion of an insertion portion having a multi-lumen tube lengthened and an intermediate portion of a coil portion provided between a coarsely wound portion and a tightly wound portion, according to a second disclosure example;

FIG. 52 is a configuration diagram of a bending portion according to a third disclosure example; and

FIG. 53 is an assembly diagram of a bending portion according to a third disclosure example.

### DETAILED DESCRIPTION OF THE PREFERRED EMBODIMENTS

Embodiments of the present invention are described below with reference to the drawings.

Note that, in each of the drawings used in the description below, the scale is different for each component in order to make each component have a recognizable size in the drawings, and the present invention is not limited only to the number of components, the shape of the components, the ratio of the sizes of the components, and the relative positional relationships of each component, shown in the drawings.

### First Embodiment

### Configuration

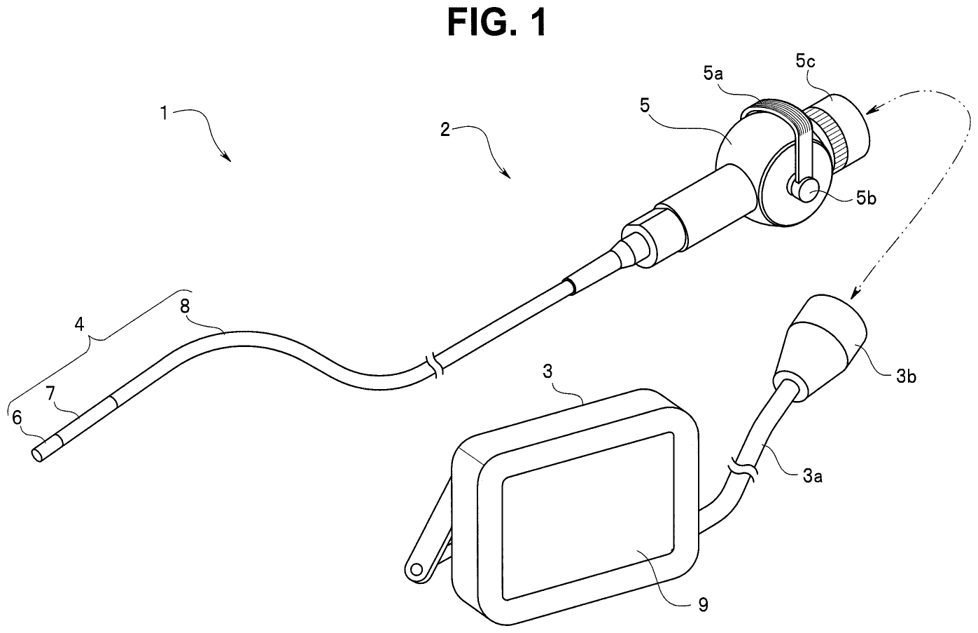

FIG. 1 is a configuration diagram of an endoscope system according to the present embodiment. As shown in FIG. 1, the endoscope system 1 includes an endoscope apparatus 2 and an apparatus main body 3 connected to the endoscope apparatus 2.

The endoscope apparatus 2 includes an elongated, small-diameter insertion portion 4 having flexibility, and an operation portion 5 connected to the proximal end portion of the insertion portion 4. The insertion portion 4 is provided with a distal end rigid portion 6, a bending portion 7, and a long flexible tube portion 8 in series in the order from the distal end side of the insertion portion 4, and the operation portion 5 is connected to the proximal end portion of the flexible tube portion 8. The distal end rigid portion 6 is provided with an observation window (not shown) and an illumination window (not shown). On the rear side of the observation window, an objective optical system that collects the reflected light from the object, an image pickup device that is an image sensor, and the like are disposed. On the rear side of the illumination window, the distal end surface of the light guide inserted into the insertion portion 4 is disposed.

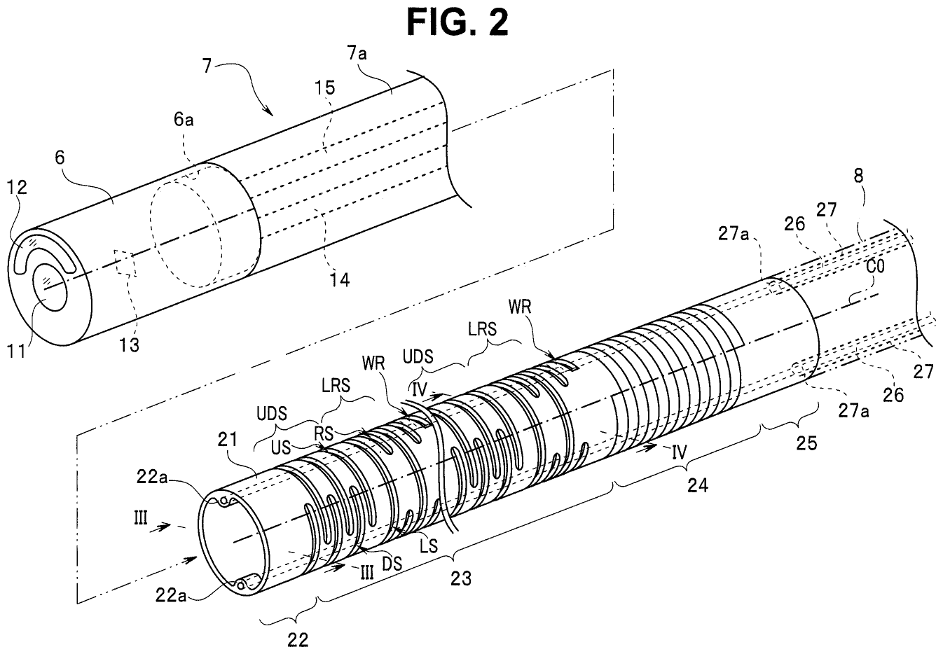

The bending portion 7 is an active bending portion that can be bent by operating the operating lever 5a provided on the operation portion 5. The bending portion of the endoscope apparatus 2 can be bent in at least one direction by at least one bending wire that is pulled or loosened in response to the operation of the operating lever 5a, and is an active bending portion that can be bent in two predetermined directions (here, two directions, up and down) in the present embodiment. The operating lever 5a can be rotated around the axis of the shaft member 5b provided on the operation portion 5, within a predetermined area. The operation portion 5 has an internal bending mechanism that bends the bending portion 7 by pulling and loosening two bending wires, which is described below, in response to the tilting operation of the operating lever 5a. In other words, the endoscope apparatus 2 has a bending portion 7 that is bent by at least one bending wire. Note that, in addition to the operating lever 5a, the operation portion 5 is also provided with various switches and the like for instructing the photographing operation of the image pickup device 13 (FIG. 2) provided in the distal end rigid portion 6.

The flexible tube portion 8 is connected to the proximal end side of the bending portion 7. The flexible tube portion 8 has a flexible tube (not shown). The flexible tube is formed by spirally winding an elongated metal thin plate around the longitudinal axis of the flexible tube portion 8. A braid that is a mesh member made of metal is provided on the outer peripheral portion of the flexible tube, and the outer peripheral portion of the braid is covered with a tube made of resin. Therefore, the flexible tube portion 8 also has appropriate flexibility, that is, softness for bending easily.

The apparatus main body 3 has a box shape, and the box-shaped exterior housing is provided with a monitor 9 for displaying an endoscope image obtained by performing image pickup of the endoscope apparatus 2. A cable 3a extends from the apparatus main body 3. A scope connecting portion 3b is provided at the distal end portion of the cable 3a. The scope connecting portion 3b is detachable from a connector 5c provided on the operation portion 5.

A processor for controlling the operation of the endoscope system 1, a drive circuit of an image pickup device, a drive circuit of a monitor 9, and the like are disposed in the apparatus main body 3. Furthermore, a light source (not shown) is built in the operation portion 5, and the light source is driven by a light source drive circuit in the apparatus main body 3. By connecting the connector 5c to the scope connecting portion 3b, the endoscope apparatus 2 is connected to the apparatus main body 3. The scope connecting portion 3b and the connector 5c each have a connector structure that is detachable from each other and enables electrical connection.

FIG. 2 is a configuration diagram of a bending portion 7 of the insertion portion 4. The distal end rigid portion 6 has a cylindrical shape and is made of a metal such as stainless steel. A circular observation window 11 and an arc-shaped illumination window 12 formed so as to surround a part of the outer edge of the observation window 11 are provided on the distal end surface of the distal end rigid portion 6. The image pickup device 13 is disposed inside the distal end rigid portion 6. An objective optical system (not shown) and an image pickup device 13 are arranged on the rear side the observation window 11. The signal cable 14 connected to the image pickup device 13 extends from the distal end rigid portion 6 in the proximal end direction. The distal end surface of the light guide 15 is disposed and fixed on the rear side of the illumination window 12.

Inside the bending portion 7, a pipe 21 which is a tubular member made of an elastic metal such as an alloy of nickel and titanium (Ni-Ti alloy) is built. The distal end portion (distal end connecting portion 22 to be described below) of the pipe 21 is fitted into a recess portion 6a on the proximal end side of the distal end rigid portion 6 and fixed to the distal end rigid portion 6 with an adhesive or a screw (not shown).

The pipe 21 has a distal end connecting portion 22, a slit portion 23, a coil portion 24, and a flexible tube portion connecting portion 25 in the order from the distal end side. The slit portion 23 and the coil portion 24 are formed by laser processing.

The distal end connecting portion 22 is a tubular portion of the pipe 21 where a slit or the like is not formed only in a predetermined area from the distal end of the pipe 21.

The slit portion 23 has a plurality of slits (that is, through grooves) each formed along the circumferential direction of the pipe 21. Here, the slit portion 23 includes: upper and lower slit portions UDS in which a plurality of (six here) slits are formed on the upper side and the lower side of the pipe 21 so as to bend in an up-down direction; and left and right slit portions LRS in which a plurality of (four here) slits are formed on the right side and the left side of the pipe 21.

In each upper and lower slit portion UDS, there are alternately formed: upper side slits US each having a slit formed on the upper side (that is, the upper bending direction side) of the pipe 21; and lower side slits DS each having a slit formed on the lower side (that is, the lower bending direction side) of the pipe 21. In other words, the slit portion 23 has a plurality of slits formed so that the bending portion 7 can bend in a predetermined bending direction.

In the left and right slit portion LRS, there are alternately formed: right side slits RS each having a slit formed on the right side (that is, the right direction side) of the pipe 21; and left side slits LS each having a slit formed on the left side (that is, the left direction side) of the pipe 21.

Note that, here, the up-down direction of the bending portion 7 coincides with the up-down direction on the endoscope screen displayed on the monitor 9, and the left-right direction of the bending portion 7 coincides with the left-right direction on the endoscope screen displayed on the monitor 9.

As shown in FIG. 2, a plurality of upper and lower slit portions UDS and left and right slit portions LRS are provided in the order from the distal end side of the pipe 21 so as to alternate with each other. A plurality of wire receiving portions WR are formed in the middle of the slit portion 23. The wire receiving portion WR is a space formed inside the pipe 21 by lance bending processing.

The coil portion 24 is provided on the proximal end side of the slit portion 23. The coil portion 24 is a tightly wound coil formed by making a spiral cut in the thin-walled portion of the pipe 21 around the central axis C0 by laser processing.

As described above, the bending portion 7 has a first region of the slit portion 23 and a second region of the coil portion 24 in the longitudinal direction of the bending portion 7.

The distal end side portion of the distal end connecting portion 22 is fitted into and fixed to the recess portion 6a formed on the proximal end side of the distal end rigid portion 6. The outer peripheral portion of the pipe 21 fixed to the distal end rigid portion 6 is covered with a resin tube 7a.

Two convex portions 22a for fixing the distal ends of the two bending wires 26 are provided on the inner peripheral surface of the distal end portion of the distal end connecting portion 22. Each bending wire 26 is inserted into a coil pipe 27 disposed and fixed in the insertion portion 4. A distal end portion 27a of each coil pipe 27 is fixed to the proximal end side of the coil portion 24. Here, the distal end portion 27a of each coil pipe 27 is fixed on the inner peripheral surface of the pipe sleeve member at the distal end portion of the flexible tube portion 8 by an adhesive or welding or the like. In other words, the two bending wires 26 are held by the two coil pipes 27 on the proximal end side of the coil portion 24.

The distal end portion 27a of each coil pipe 27 is fixed on the proximal end side of the coil portion 24 (here, inside the flexible tube portion 8), and the two coil pipes 27 are not provided in the bending portion 7. As a result, in the coil portion 24, the number of internal components in the bending portion 7 is reduced by two (the two coil pipes 27), so that the small-diameter bending portion 7 is easily bent.

Note that the distal end portion 27a of each coil pipe 27 may be fixed on the inner peripheral surface of the flexible tube portion connecting portion 25 by an adhesive or welding or the like.

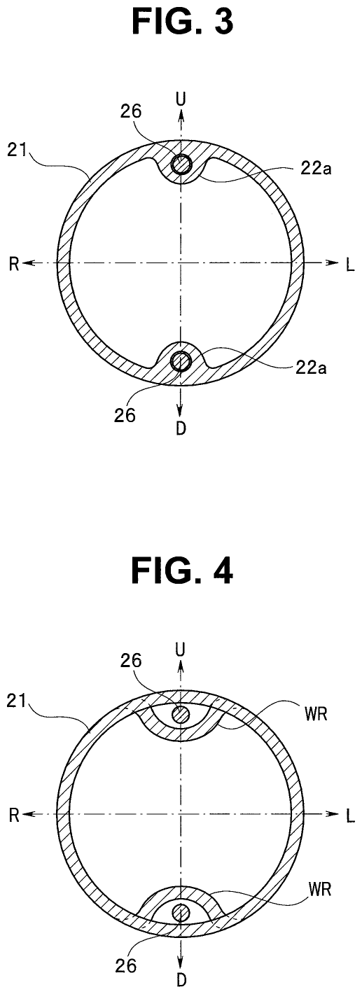

FIG. 3 is a cross-sectional view of the distal end connecting portion 22 of the pipe 21 when the pipe 21 is viewed from the distal end side. FIG. 3 shows a cross section of the pipe 21 along lines of FIG. 2. Two convex portions 22a are formed on the inner peripheral surface on the distal end side of the pipe 21 at two positions facing each other in up-down bending direction (UD) of the bending portion 7. As shown in FIG. 3, a hole is formed in each convex portion 22a, and the distal end of the bending wires 26 is inserted into the hole and fixed to the hole by soldering or welding.

Note that the distal end portion convex portion 22a of the distal end connecting portion 22 has a shape that is integrally processed in FIG. 3, and is, for example, a shaped portion formed by providing an internal convex portion 22a on a pipe material, which is subjected to machining or the like on the inner surface of the pipe material in the case. Alternatively, the distal end connecting portion 22 may be a pipe member fixed to the distal end connecting portion by welding or the like, or may be formed so as to insert and fix the bending wires 26 through the internally bent portions each having the same shape as the wire receiving portion WR in the bending portion.

FIG. 4 is a cross-sectional view of a portion in which the two wire receivers WR of the pipe 21 are formed when the pipe 21 is viewed from the distal end side. FIG. 4 shows a cross section of the pipe 21 along the IV-IV line of FIG. 2. Two wire receivers WR are formed on the inner peripheral surface of the pipe 21 at two positions facing each other in up-down bending direction of the bending portion 7. As shown in FIG. 4, one of the two bending wires 26 is inserted into a plurality of wire receivers WR provided on the inner peripheral surface of the pipe 21 in the upward bending direction, and the other one of the two bending wires 26 is inserted into a plurality of wire receivers WR provided on the inner peripheral surface of the pipe 21 in the downward bending direction.

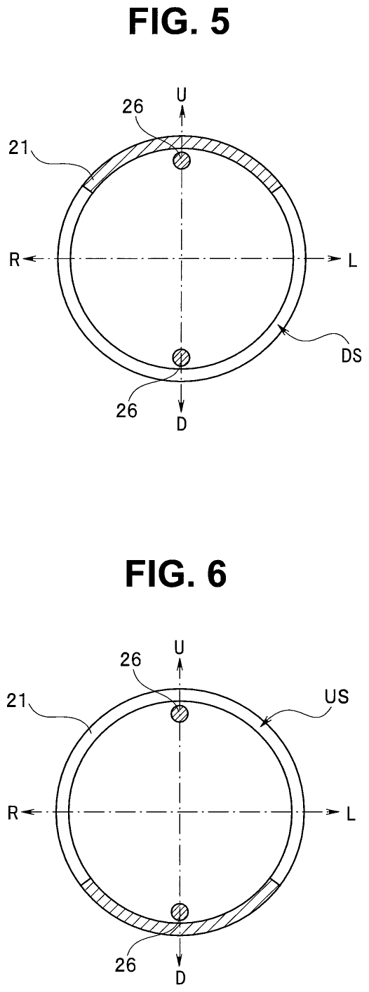

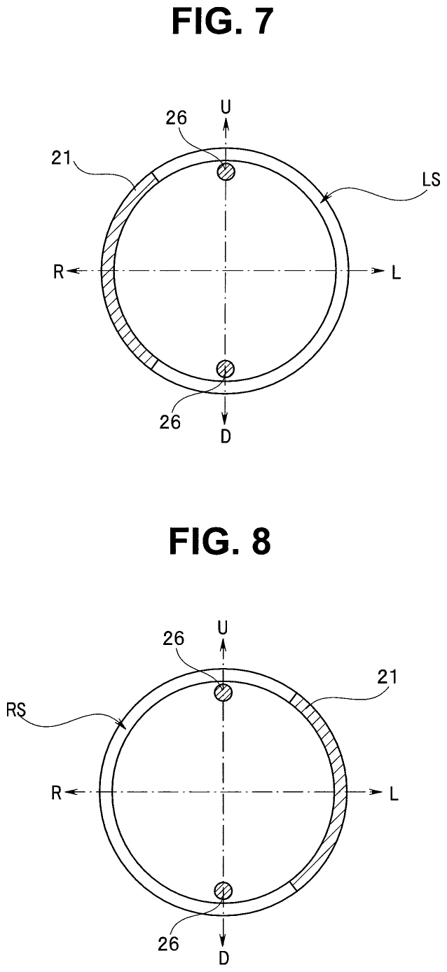

FIG. 5 is a cross-sectional view of a portion in which a lower side slit DS of the pipe 21 is formed when the pipe 21 is viewed from the distal end side. FIG. 6 is a cross-sectional view of a portion in which an upper side slit US of the pipe 21 is formed when the pipe 21 is viewed from the distal end side. FIG. 7 is a cross-sectional view of a portion in which a left side slits LS of the pipe 21 is formed when the pipe 21 is viewed from the distal end side. FIG. 8 is a cross-sectional view of a portion in which a right side slit RS of the pipe 21 is formed when the pipe 21 is viewed from the distal end side.

As shown in FIGS. 5 to 8, the individual lower side slits DS, the individual upper side slits US, the individual left side slits LS, and the individual right side slits RS are through grooves each having a length longer than the half circumference of a circle in a cross section orthogonal to the longitudinal axis of the pipe 21 in the circumferential direction of the pipe 21.

Pulling or loosening the bending wire 26 widens or narrows the width of each slit, so that the slit portion 23 is bent.

The region of the coil portion 24 is a region of the bending portion 7 that bends following the bend of the slit portion 23. In other words, the region of the coil portion 24 is provided on the proximal end side of the region of the slit portion 23, and bends following the bend of the slit portion 23. For example, when a force is applied to the distal end of the bending portion 7 from the outside, the distal end side of the bending portion 7 is bent and the coil portion 24 on the proximal end side is bent.

Note that, in the above-mentioned example, each slit of the slit portion 23 and the tightly wound coil of the coil portion 24 are formed by processing one pipe 21. More specifically, laser processing forms a plurality of slits in one pipe 21 and spirally cuts a thin-walled portion of the pipe 21 around the central axis C0, so that the slit portion 23 and the coil portion 24 are integrally formed. However, the coil portion 24 and the slit portion 23 each may be separately produced, and the distal end portion of the coil portion may be connected to the proximal end side of the slit portion 23 by welding, so that the coil portion 24 and the slit portion 23 may be integrally formed.

Note that, in the above-described description, the coil portion is described as a tightly wound coil, but processing by a laser, wire cutting, or the like generates a slight gap. The gap is made as small as possible to be a possible minimum compared to the slit portion 23. It is conceivable to make the gap as small as possible with a high aspect ratio processing machine, or to process the surface into a coil shape by laser processing and then apply a stress to the pipe to cut the pipe into the coil shape. Furthermore, it is conceivable to perform heat treatment after processing to re-form the shape so that there are no gaps. Alternatively, there is also a method in which the coil portion 24 is made into a close contact coil by processing the wire rod into a coil, and the coil portion 24 is integrated with the slit portion 23 by welding or the like.

The flexible tube portion connecting portion 25 provided on the proximal end side of the coil portion 24 is a portion to which the distal end portion of the flexible tube portion 8 is connected. The flexible tube portion 8 is fitted into the cylindrical flexible tube portion connecting portion 25 and fixed by an adhesive.

As described above, the distal end side portion of the resin tube 7a covers the outer peripheral portion of the distal end connecting portion 22 fixed to the distal end rigid portion 6, and the proximal end side portion of the resin tube 7a (not shown) also covers the outer peripheral portion of the flexible tube portion connecting portion 25. The distal end side portion and the proximal end side portion of the resin tube 7a are thread-wound (not shown), coated with an adhesive, and fixed to the distal end connecting portion 22 and the flexible tube portion connecting portion 25.

### Actions

First, before description is made on actions of the insertion portion of the above-described embodiment, description is made below on actions of the endoscope insertion portion having the structure of a comparative example.

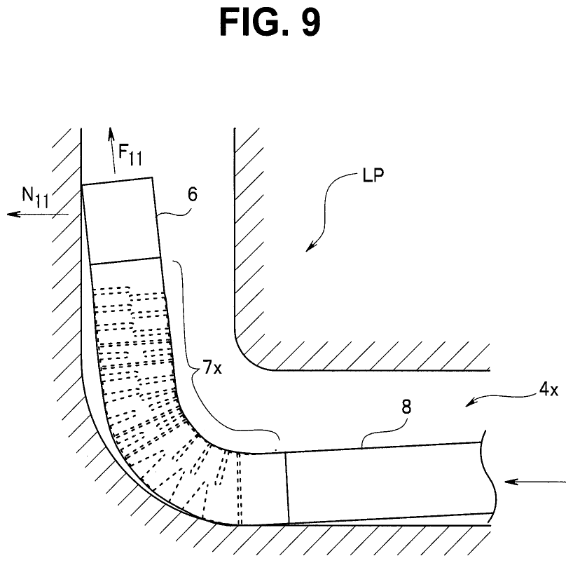

FIG. 9 is a diagram for explaining an action when the distal end portion of an insertion portion 4x of an endoscope having a structure of a comparative example is pushed into an L-shaped pipe LP. The insertion portion 4x of the comparative example has a distal end rigid portion 6, a bending portion 7x, and a flexible tube portion 8 in this order from the distal end side. The bending portion 7x includes the slit portion 23 described above, but has a structure in which the coil portion 24 is not provided on the proximal end side of the slit portion 23.

When the user pushes the distal end rigid portion 6 of the insertion portion 4x into the pipe LP while the user grips the flexible tube portion 8 of the insertion portion 4 and, as shown in FIG. 9, the distal end rigid portion 6 turns the elbow portion of the pipe LP and further advances, one or more slits are subjected to a stress to greatly expand the slits in a part of the proximal end side portion of the bending portion 7x, so that a part of the slits in the slit portion 23 may crack and rupture.

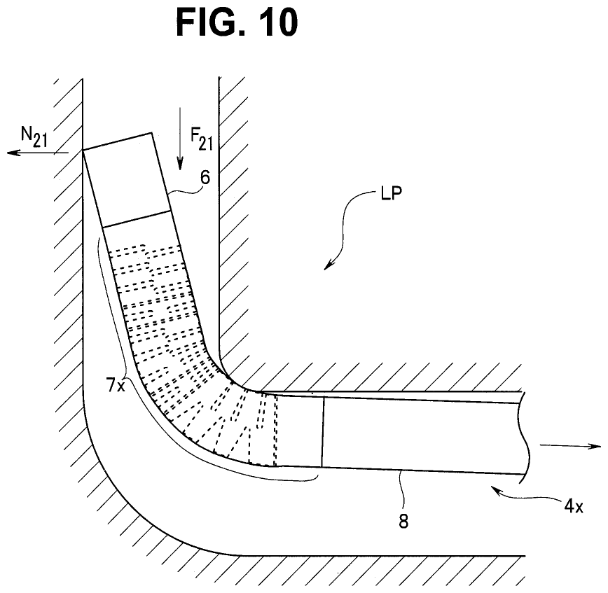

FIG. 10 is a diagram for explaining an action when the insertion portion 4x of the endoscope having the structure of the comparative example is pulled out from the L-shaped pipe LP. When the user pulls out the distal end rigid portion 6 of the insertion portion 4x from the pipe LP while the user grips the flexible tube portion 8 of the insertion portion 4x and, as shown in FIG. 10, the bending portion 7x passes through the elbow portion of the pipe LP, the proximal end side portion of the bending portion 7x comes into contact with the projecting part of the inner wall surface of the pipe LP. When the proximal end side portion of the bending portion 7x comes into contact with the projecting part, one or more slits in the slit portion 23 on the opposite side of the contact portion is greatly expanded so that a part of the slits of the slit portion 23 may crack and rupture.

Description is made below on actions of the insertion portion of the endoscope of the above-described embodiments in contrast to the endoscope having the structure of the above-mentioned comparative example.

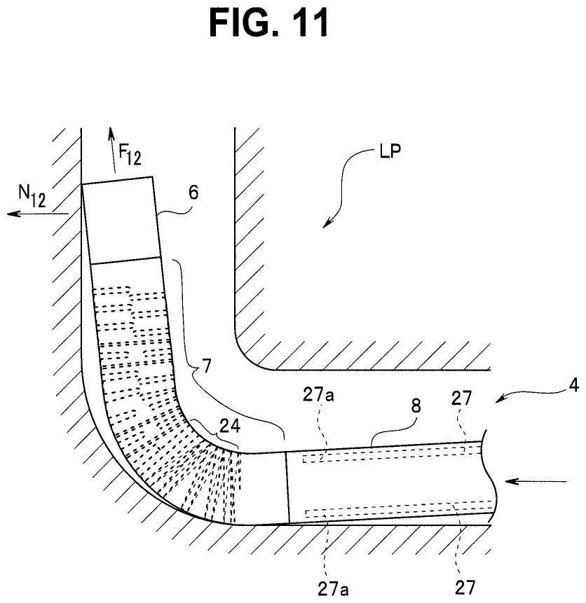

FIG. 11 is a diagram for explaining an action when the distal end portion of the insertion portion 4 of the endoscope having a structure of the embodiment is pushed into the L-shaped pipe LP. As shown in FIG. 11, when the distal end rigid portion 6 turns the elbow portion of the pipe LP and then further advances, the coil portion 24 at the proximal end side portion of the bending portion 7 is greatly deformed, which reduces the stress applied to one or more slits of the slit portion 23. As a result, it is possible to prevent the slit portion 23 of the bending portion 7 from being damaged.

In the distal end rigid portion 6 of the comparative example in FIG. 9, let N₁₁ be the force applied to the inner wall of the pipe LP and let F₁₁ be the force for traveling in the distal end direction, and in the distal end rigid portion 6 of the embodiment in FIG. 11, let N₁₂ be the force applied to the inner wall of the pipe LP and let F₁₂ be the force for traveling in the distal end direction, and then N₁₁>N₁₂ due to the deformation of the coil portion 24. As a result, F₁₁<F₁₂, and there is also an effect that the user can easily insert the insertion portion 4.

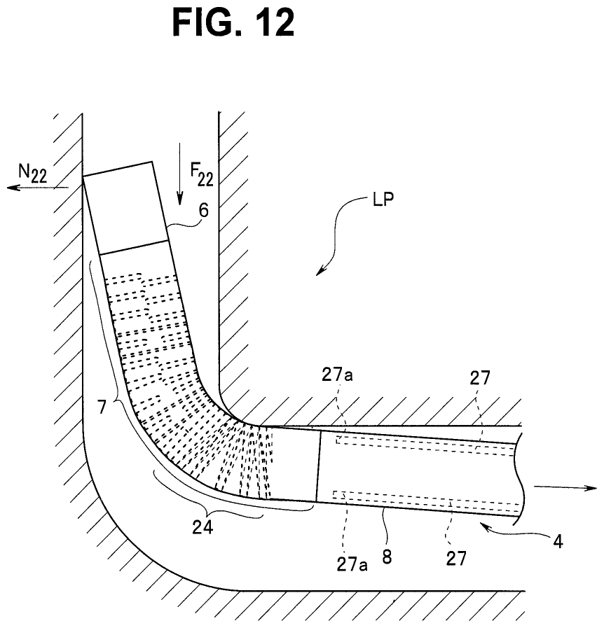

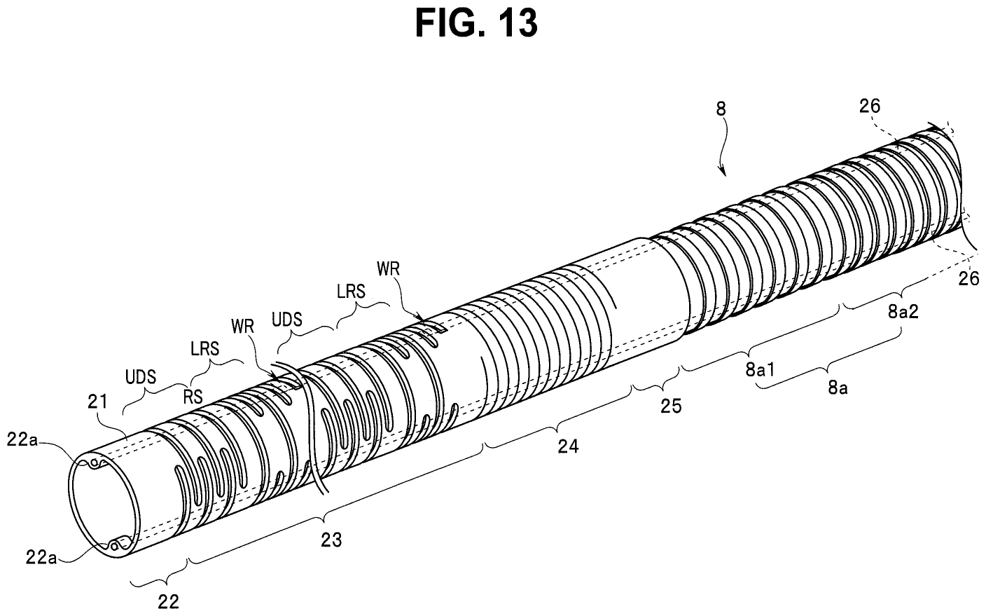

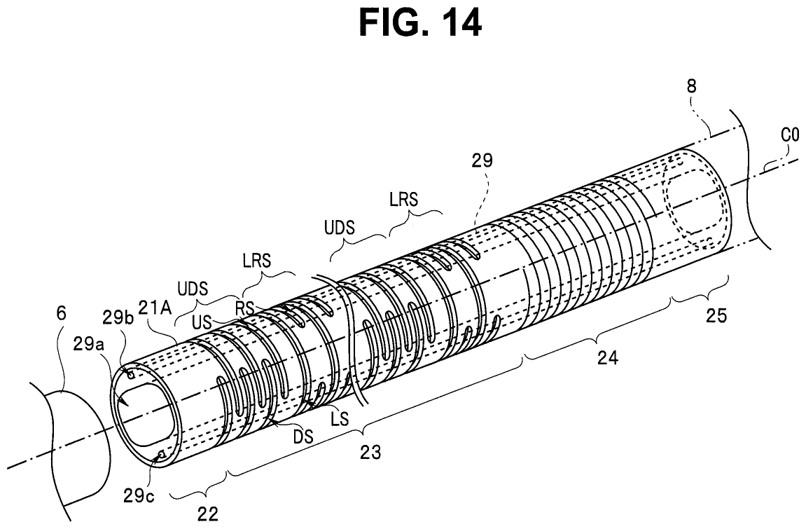

FIG. 12 is a diagram for explaining an action when the distal end portion of the insertion portion 4 of the endoscope having the structure of the embodiment is pulled out from the L-shaped pipe LP. As shown in FIG. 12, when the distal end rigid portion 6 approaches the elbow portion of the pipe LP and then is further pulled out, the coil portion 24 at the proximal end side portion of the bending portion 7 is greatly deformed, which reduces the stress applied to one or more slits of the slit portion 23. As a result, it is possible to prevent the slit portion 23 of the bending portion 7 from being damaged.

In the distal end rigid portion 6 of the comparative example in FIG. 10, let N₂₁ be the force applied to the inner wall of the pipe LP and let F₂₁ be the force for traveling toward the proximal end direction, and in the distal end rigid portion 6 of the embodiment in FIG. 12, let N₂₂ be the force applied to the inner wall of the pipe LP and let F₂₂ be the force for traveling in the proximal end direction, and then N₂₁>N₂₂ due to the deformation of the coil portion 24. As a result, F₂₁<F₂₂, and there is also an effect that the user can easily pull out the insertion portion 4.

As described above, according to the above-described embodiment, it is possible to provide an endoscope apparatus and a bending member for endoscope in which the bending portion of the insertion portion is hard to break.

### Modification 1

In the first embodiment described above, the flexible tube portion 8 connected to the proximal end of the bending portion 7 has the same flexibility over the entire longitudinal direction, but the distal end side portion (that is, the portion adjacent to the bending portion 7) of the flexible tube portion 8 may be softened so as to be more easily bent than the proximal end side portion.

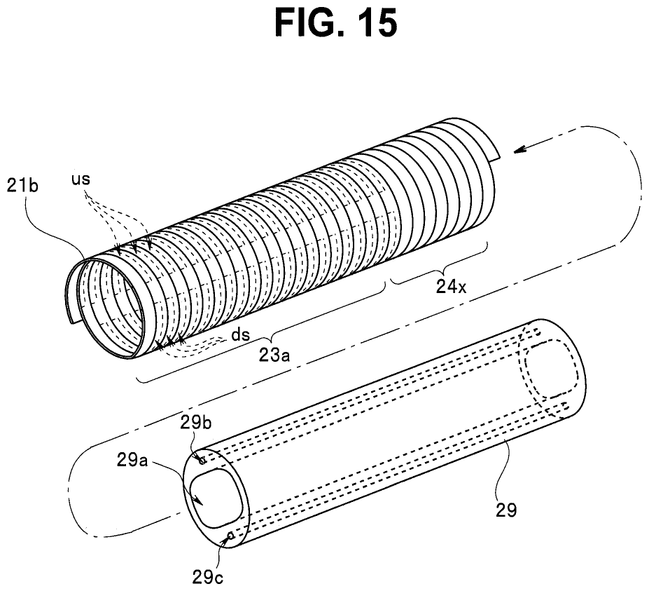

FIG. 13 is a configuration diagram of a bending portion 7 and a flexible tube portion 8 of the insertion portion according to a modification 1 of the first embodiment. FIG. 13 shows the pipe 21 in the bending portion 7 and the flexible tube 8a in the flexible tube portion 8, but omits to show the resin tube 7a that covers the bending portion 7, the outer skin that covers the outer peripheral surface of the flexible tube portion 8, and the like.

The flexible tube portion 8 includes an outer skin made of resin (not shown), a metal mesh braid (not shown), and a flexible tube 8a around which an elongated, thin, metal plate member is spirally wound. More specifically, the flexible tube portion 8 includes a flexible tube 8a into which an internal component such as a signal cable 14 is inserted, a braid (not shown) covering the flexible tube 8a, and a tubular outer skin covering the braid (not shown).

As shown in FIG. 13, a flexible tube 8a, which is made of metal and is a thin plate-shaped member, is wound around the internal component in the flexible tube portion 8. In the modification, the winding pitch at the distal end side portion 8a1 of the flexible tube 8a is made greater than the winding pitch at 8a2 that is a portion other than the distal end side portion 8a1 of the flexible tube portion 8, so that the distal end side portion 8a1 of the flexible tube portion 8 is made easier to bend than the other portion 8a2. In other words, the pitch width when the thin plate-shaped member is wound in the distal end side portion 8a1 of the flexible tube 8a is greater than the pitch width when the thin plate-shaped member is wound in the other portion 8a2, so that the Young's modulus of the distal end side portion 8a1 is smaller than the Young's modulus of the other portion 8a2. Namely, the distal end side portion 8a1 is more easily bent than the other portion 8a2 with respect to bending stress.

Let Y1 be the Young's modulus of the coil portion 24, let Y2 be the Young's modulus of the distal end side portion 8a1, and let Y3 be the Young's modulus of the other portion 8a2, and then the following expression (1) holds.

Y1≤Y2<Y3 (1)

Since the Young's modulus Y2 of the distal end side portion 8a1 of the flexible tube portion 8 is equal to or greater than the Young's modulus Y1 of the coil portion 24 of the bending portion 7 and smaller than the Young's modulus Y3 of the other portion 8a2 of the flexible tube portion 8, the distal end side portion 8a1 is more easily bent than the other portion 8a2 when the coil portion 24 is bent. Therefore, when stress is applied to the bending portion 7, the distal end side portion 8a1 of the flexible tube portion 8 is more easily bent than the other portion 8a2, so that the distal end side portion 8a1 can absorb a part of the stress applied to the bending portion 7 to reduce the stress applied to the bending portion 7.

Note that the relationship between the three Young's modulus may have the relationship represented by the following expression (2).

Y1>Y2, and Y1<Y3 (2)

The same effect can also be obtained if the condition of the expression (2) is satisfied.

As described above, the distal end side portion 8a, adjacent to the bending portion 7, of the flexible tube portion 8 has a smaller Young's modulus than the other portion 8a2, which is other than the portion 8a.

The insertion portion having the bending portion of the modification 1 also produces the same effect as the effect of the first embodiment.

### Modification 2

In the first embodiment described above, the internal component is directly inserted into the pipe 21 of the bending portion 7, and the two bending wires 26 are inserted into a plurality of wire receivers WR, but a multi-lumen tube may be inserted into the pipe so that the internal component and the two bending wires may be inserted into a plurality of holes formed in the multi-lumen tube.

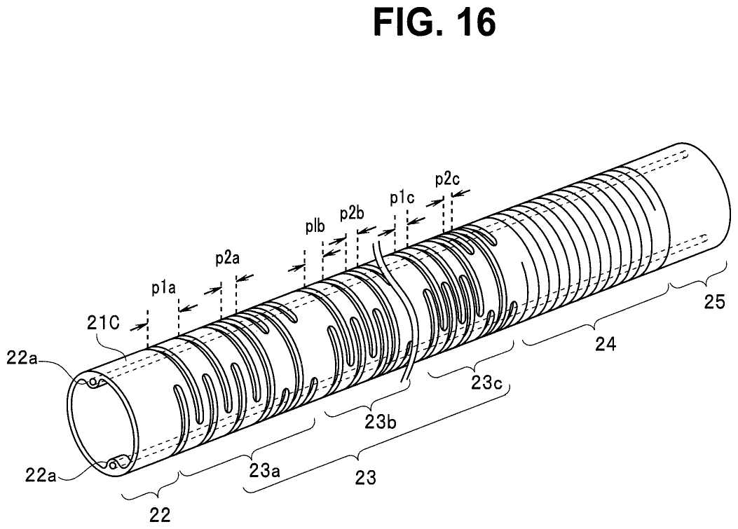

FIG. 14 is a configuration diagram of a bending portion 7 of the insertion portion according to a modification 2 of the first embodiment. FIG. 14 shows a pipe 21A in the bending portion 7 and a multi-lumen tube 29 inserted in the pipe 21A.

The multi-lumen tube 29 is made of a soft resin such as silicone. The multi-lumen tube 29 is formed with three holes 29a, 29b and 29c. Of the three holes, the hole 29a along the central axis is a hole into which an internal component such as a signal cable 14 is inserted. The other two holes 29b and 29c of the three holes are holes into which the two bending wires 26 are inserted. Therefore, the pipe 21A does not need to be provided with a plurality of wire receivers WR, and has a simple configuration. Alternatively, the pipe 21A may be formed of a superelastic alloy.

As described above, the bending portion 7 has a cylindrical pipe 21A including the slit portion 23 and the coil portion 24. Furthermore, the bending portion 7 has a multi-lumen tube 29 inserted into the pipe 21A, and the two bending wires 26 are inserted in two holes 29b formed in the multi-lumen tube 29.

The insertion portion having the bending portion of the modification 2 also produces the same effect as the effect of the first embodiment.

### Modification 3

In the first embodiment described above, each slit of the upper and lower slit portions UDS is a through groove formed in the circumferential direction with respect to the cylindrical pipe 21, but a tightly wound coil may be processed to form the upper and lower slit portions.

FIG. 15 is a configuration diagram of a bending portion 7 of the insertion portion according to a modification 3 of the first embodiment. For example, laser processing is applied to a tightly wound coil 21b formed so that an elongated plate-shaped member winds around the central axis, to form a plurality of upper side slits us on the upper portion in the bending direction at predetermined intervals along the central axis in the longitudinal direction of the coil 21b as shown by the dotted line. Similarly, laser processing is applied to the coil 21b to form a plurality of lower side slits ds on the lower portion in the bending direction at predetermined intervals along the central axis in the longitudinal direction of the coil 21b as shown by the dotted line.

The plurality of upper side slits us and the plurality of lower side slits ds are formed in the distal end side portion of the coil 21b, and are not formed in the proximal end side portion 24x of the coil 21b. Note that the wire receiving portions WR are formed at a plurality of positions (not shown).

The bending portion 7 using such a coil 21b also produces the same effect as the effect of the bending portion 7 of the first embodiment.

Note that the multi-lumen tube 29 shown in the above-described modification 2 may be further inserted into the coil 21b. The multi-lumen tube 29 is formed with three holes 29a, 29b and 29c. Of the three holes, the hole 29a along the central axis is a hole into which an internal component is inserted. The other two holes 29b and 29c of the three holes are holes into which the two bending wires 26 are inserted. Therefore, the pipe 21A does not need to be provided with a plurality of wire receivers WR.

### Modification 4

In the first embodiment described above, the distances between the upper side slits US and between the lower side slits DS in the upper and lower slit portions UDS in the pipe 21 of the bending portion 7, that is, the pitches are constant, and the distances between the right side slits RS and between the left side slits LS in the left and right slit portions LRS, that is, the pitches are also constant, but respective pitches may be changed so that the pitches decreases from the distal end side toward the proximal end side.

FIG. 16 is a configuration diagram of a bending portion 7 of the insertion portion according to a modification 4 of the first embodiment. As shown in FIG. 16, pitches p1a between the upper side slits US and between the lower side slits DS in the distalmost slit portion 23a of the pipe 21C are greater than pitches p1b between the upper side slits US and between the lower side slits DS in the proximal end side slit portion 23b adjacent to the distalmost slit portion 23a. Similarly, pitches p2a between the right side slits RS and between the left side slits LS in the distalmost slit portion 23a of the pipe 21C are greater than pitches p2b between the right side slits RS and between the left side slits LS in the proximal end side slit portion 23b adjacent to the distalmost slit portion 23a.

Similarly, pitches p1c between the upper side slits US and between the lower side slits DS in the proximalmost slit portion 23c of the pipe 21C are smaller than the pitches between the upper side slits US and between the lower side slits DS in the slit portion 23 on the distal end side of the proximalmost slit portion 23c. Similarly, the pitches p3c between the right side slits RS and between the left side slits LS in the proximalmost slit portion 23c of the pipe 21C are smaller than the pitches between the right side slits RS and between the left side slits LS in the slit portion 23 on the distal end side of the proximalmost slit portion 23c.

As described above, the slit portion 23 includes, in the longitudinal direction of the bending portion 7, parts in which pitches between two adjacent slits are different, so that the slit portion 23 is formed so as to increase the bendability from the coil portion 24 toward the distal end of the slit portion 23.

Thus, the pitches between the upper side slits US, the pitches between the lower side slits DS, the pitches between the right side slits RS, and the pitches between the left side slits LS decrease from the distal end side to the proximal end side, so that distal end side can be bent more easily.

### Modification 5

In the first embodiment described above, the lengths of the upper side slits US and the lower side slits DS of the upper and lower slit portion UDS in the pipe 21 of the bending portion 7 are constant in the circumferential direction, and the lengths of the right side slits RS and the left side slits LS in the left and right slit portion LRS are also constant in the circumferential direction, but the lengths of respective slits in the circumferential direction may decrease from the distal end side toward the proximal end side.

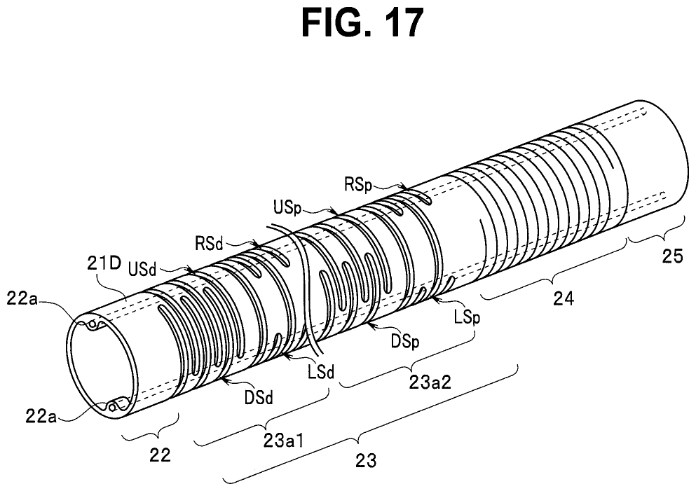

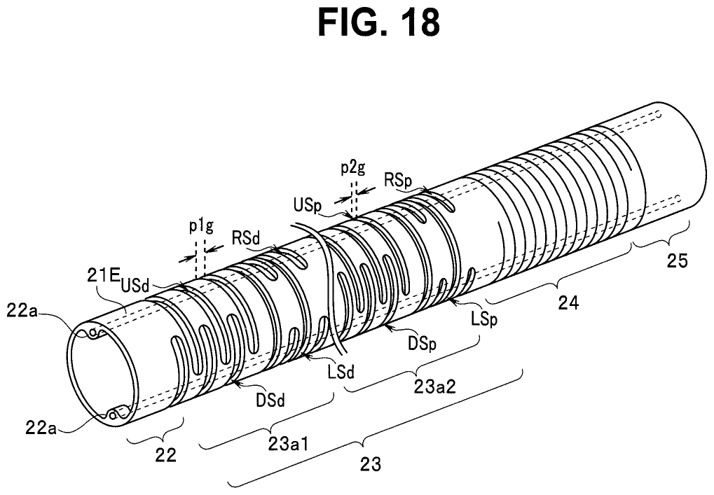

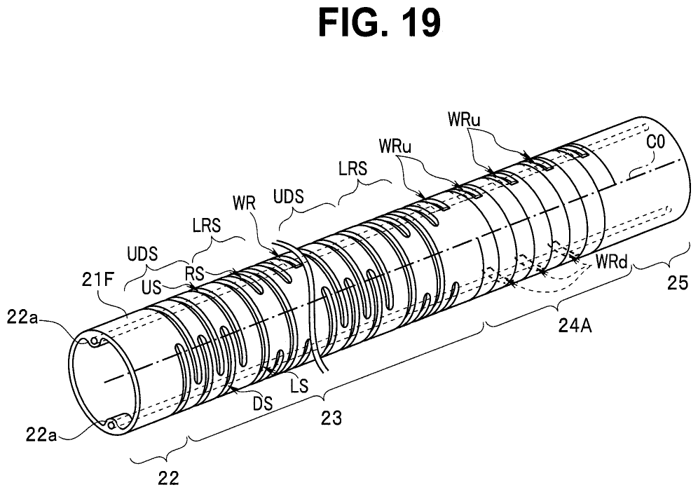

FIG. 17 is a configuration diagram of a bending portion 7 of the insertion portion according to a modification 5 of the first embodiment. As shown in FIG. 17, the lengths in the circumferential direction of upper side slits USd and lower side slits DSd in the distalmost slit portion 23a1 of the pipe 21D is longer than the length in the circumferential direction of the upper side slits US and the lower side slits DS in the proximal end side slit portion 23 adjacent to the distalmost slit portion 23a1. Similarly, each slit is formed such that, in two adjacent slit portions 23, the length in the circumferential direction of each slit of the slit portion 23 on the distal end side is longer than the length in the circumferential direction of each slit of the slit portion 23 on the proximal end side.

Then, each of the lengths in the circumferential direction of the upper side slits USp and the lower side slits DSp in the proximalmost slit portion 23a2 of the pipe 21D is shorter than any of the lengths in the circumferential direction of the upper side slits US and the lower side slits DS in all the slit portions 23 on the distal end side of the proximalmost slit portion 23a2. Similarly, each of the lengths in the circumferential direction of the right side slits RSp and the left side slits LSp in the proximalmost slit portion 23a2 of the pipe 21D is shorter than any of the lengths in the circumferential direction of the right side slits RS and the left side slits LS in all the slit portions 23 on distal end side of the proximalmost slit portion 23a2.

As described above, the slit portion 23 includes, in the longitudinal direction of the bending portion 7, parts in which the lengths of the slits in the circumferential direction of the bending portion 7 are different, so that the slit portion 23 is formed so as to increase the bendability from the coil portion 24 toward the distal end of the slit portion 23.

Thus, reducing the lengths in the circumferential direction of the upper side slits US and the lower side slits DS, and the lengths in the circumferential direction of the right side slits RS and the left side slits LS from the distal end side toward the proximal end side makes the distal end side easier to bend.

### Modification 6

In the first embodiment described above, the upper side slits US and the lower side slits DS in the upper and lower slit portions UDS in the pipe 21 of the bending portion 7 each have a constant length in the direction of the central axis C0 of the bending portion 7, that is, a constant groove width. The right side slits RS and the left side slits LS in the left and right slit portions LRS each also have a constant length in the central axis C0 direction, that is, a constant groove width. However, the lengths of the slits may be changed so that the respective lengths in the central axis C0 direction decrease, that is, the respective groove widths decrease, from the distal end side to the proximal end side.

FIG. 18 is a configuration diagram of a bending portion 7 of the insertion portion according to a modification 6 of the first embodiment. As shown in FIG. 18, each length (groove width) p1g in the longitudinal direction (central axis C0 direction) of the bending portion 7 of the upper side slits USd, the lower side slits DSd, the right side slits RSd and the left side slits LSd in the distalmost slit portion 23a1 of the pipe 21E is longer than each length (groove width) in the longitudinal direction (central axis C0 direction) of the bending portion 7 of the upper side slits US, lower side slits DS, right side slits RS and left side slits LS in the slit portion 23 on the proximal end side adjacent to the distalmost slit portion 23a1 (the adjacent portion is not shown due to the break line).

Similarly, a plurality of the slits are formed so that the width of the slit on the proximal end side is narrower than the width of the slit on the distal end side.

Then, each length (groove width) p2g in the longitudinal direction (central axis C0 direction) of the bending portion 7 of the upper side slits USp, the lower side slits DSp, the right side slits RSp and the left side slits LSp in the proximalmost slit portion 23a2 of the pipe 21E is shorter than any length (groove width) (p1g, . . . ), in the longitudinal direction (central axis C0 direction) of the bending portion 7, of the upper side slits US, the lower side slits DS, the right side slits RS and the left side slits LS in all the slit portions 23 (23a, . . . ) on the distal end side of the proximalmost slit portion 23a2.

As described above, the slit portion 23 includes, in the longitudinal direction of the bending portion 7, parts in which the widths of the plurality of slits are different, so that the slit portion 23 is formed so as to increase the bendability from the coil portion 24 toward the distal end of the slit portion 23.

As described above, shortening (narrowing) the length (groove width) in the longitudinal direction (central axis C0 direction) of the bending portion 7 of the upper side slits US, the lower side slits DS, the right side slits RS, and the left side slits LS, from the distal end side toward the proximal end side makes the distal end side bend more easily.

### Modification 7

In the first embodiment described above, wire receiving portions WR are provided at a plurality of positions in the middle of the slit portion 23, but wire receiving portions WR may also be provided at one or more positions in the coil portion 24.

FIG. 19 is a configuration diagram of a bending portion 7 of the insertion portion according to a modification 7 of the first embodiment. The coil portion 24A is formed of a plate-shaped member with a wide coil width (set wider than the distance between the upper side slits US and between the lower side slits DS) on which a wire receiving portion can be formed. As shown in FIG. 19, a plurality of wire receiving portions WRu are disposed on the upward side of the coil portion 24A on the proximal end side of the pipe 21F at predetermined intervals in the longitudinal direction of the bending portion 7. Similarly, a plurality of wire receiving portions WRd are disposed on the downward side of the coil portion 24A at predetermined intervals in the longitudinal direction of the bending portion 7. The two adjacent wire receiving portions WRu are disposed every other wound plate-shaped coil, and similarly two adjacent wire receiving portions WRd are also disposed every other wound plate-shaped coil.

Thus, the coil portion 24A is also provided with a plurality of wire receiving portions WRu and WRd, so that each bending wire 26 is firmly held, in other words, supported at the coil portion 24A and the coil of the coil portion 24A is unlikely to shift in the radial direction.

In addition, making the width of the coil wider than the distance between the slits can reduce the shift between the coils against compression and bending.

### Modification 8

In the first embodiment described above, the bending portion 7 is covered with a resin tube, but there may be further provided a sheath member covering the slit portion 23 and the coil portion 24 and having a proximal end side portion harder than the distal end side portion.

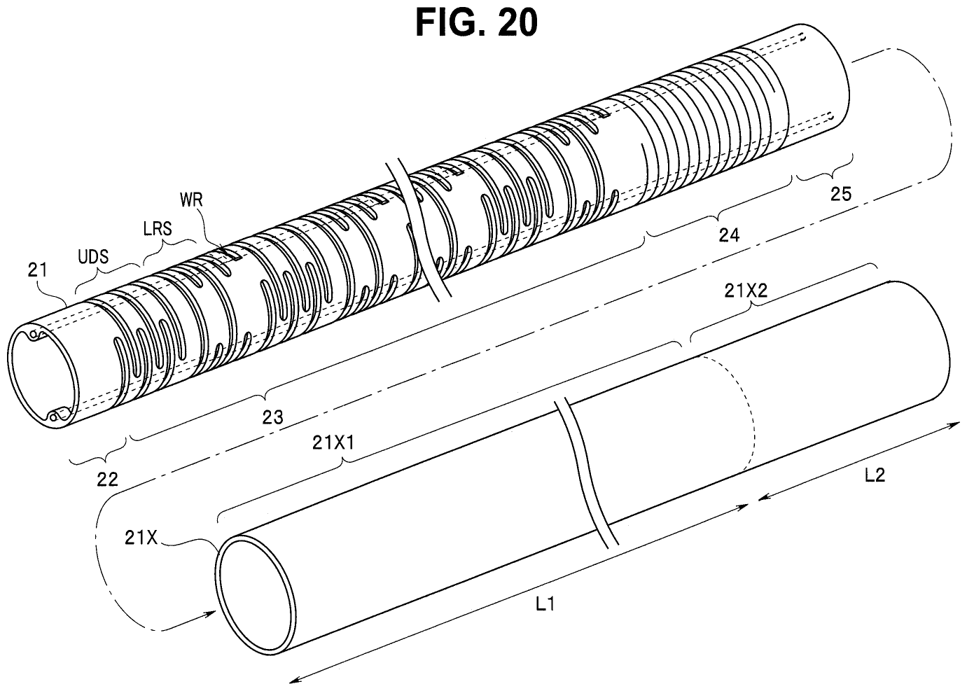

FIG. 20 is a configuration diagram of a bending portion 7 of the insertion portion according to a modification 8 of the first embodiment. The sheath member 21X is an elastic member having a cylindrical shape, and is made of rubber, for example. The sheath member 21X has substantially the same length as the pipe 21. The length L1 of the distal end side portion 21X1 of the sheath member 21X is the total length of the distal end connecting portion 22 and the slit portion 23, and the length L2 of the proximal end side portion 21X2 is the total length of the coil portion 24 and the flexible tube portion connecting portion 25.

The hardness of the distal end side portion 21X1 of the sheath member 21X is smaller than the hardness of the proximal end side portion 21X2. In other words, the sheath member 21X is a member that covers the pipe 21 of the bending portion 7, and the portion that covers a plurality of the slits has a smaller Young's modulus than the portion that covers the coil portion 24 of the tightly wound coil. Therefore, the proximal end side portion 21X2 is harder to bend than the distal end side portion 21X1.

As described above, the proximal end side portion 21X2 of the sheath member 21X that covers the coil portion 24 and the flexible tube portion connecting portion 25xB is harder than the distal end side portion 21X1 that covers the slit portion 23, so that the coil of the coil portion 24 is unlikely to shift in the radial direction when the bending portion 7 is bent.

### Modification 9

In the first embodiment described above, the thickness of the thin-walled portion of the pipe 21 is constant throughout, but the thickness of the thin-walled portion of the coil portion 24 may be made thicker than the thickness of the thin-walled portion of the slit portion 23.

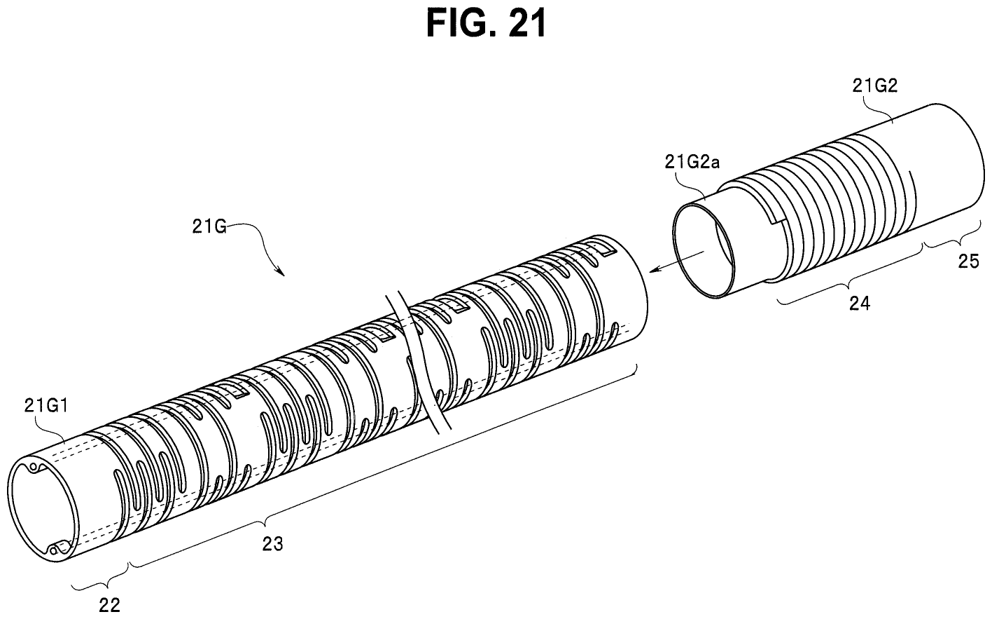

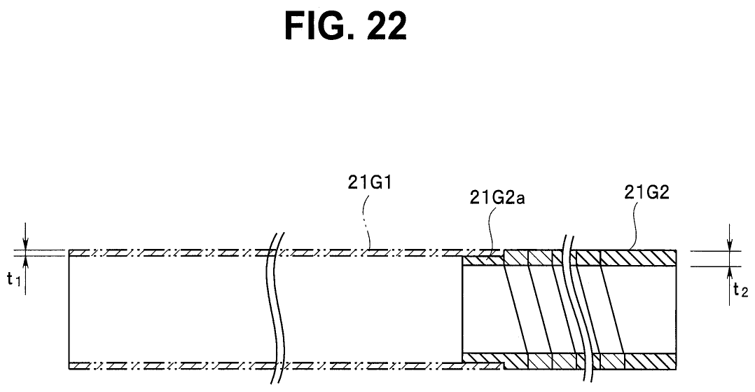

FIG. 21 is a configuration diagram of a bending portion 7 of the insertion portion according to a modification 9 of the first embodiment. FIG. 22 is a cross-sectional view of a pipe 21G of the bending portion 7 of the insertion portion 4 according to the modification 9 of the first embodiment. The pipe 21G of the bending portion 7 is configured with a distal end side portion 21G1 including the distal end connecting portion 22 and a proximal end side portion 21G2 including the flexible tube portion connecting portion 25, but the member of the proximal end side portion 21G2 including the coil portion 24 and the member of the distal end side portion 21G1 including the slit portion 23 are separate members. A thickness t1 of the distal end side portion 21G1 is smaller than a thickness t2 of the proximal end side portion 21G2. The thickness t1 is the thickness of the thin-walled portion of the distal end side portion 21G1 of the slit portion 23, and the thickness t2 is the thickness of the thin-walled portion of the proximal end side portion 21G2 of the coil portion 24. The distal end side of the proximal end side portion 21G2 has a stepped portion 21G2a that fits into the distal end side portion 21G1.

The stepped portion 21G2a is inserted from the proximal end side of the distal end side portion 21G1, and the distal end side portion 21G1 and the proximal end side portion 21G2 are then connected and fixed by welding or the like.

As described above, a plurality of the slits of the slit portion 23 are formed in the distal end side portion 21G1 which is a pipe member, and the thickness of the thin-walled portion of the tightly wound coil portion 24 is thicker than the thickness of the thin-walled portion of the distal end side portion 21G1.

Also with such a configuration, the proximal end side portion 21G2 is harder than the distal end side portion 21G1, so that the coil of the coil portion 24 is unlikely to shift in the radial direction when the bending portion 7 is bent.

Note that the pipe 21G is configured with two members in FIGS. 21 and 22, but the pipe 21G may be configured with one member.

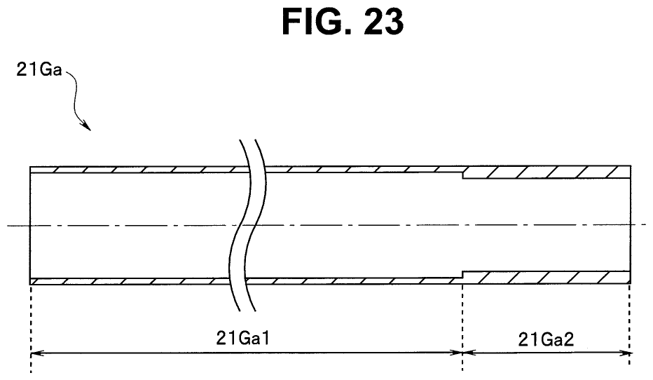

FIG. 23 is a cross-sectional view of the pipe 21Ga before the slits and the like are formed. Cutting the inner peripheral surface on the distal end side of one pipe member can reduce the thickness of the thin-walled portion of the distal end side portion 21Ga1. The distal end side portion 21Ga1 serves as a distal end connecting portion 22 and a slit portion 23. The proximal end side portion 21Ga2 serves as a coil portion 24 and a flexible tube portion connecting portion 25.

The respective slits of the slit portion 23 and the wire receiving portion WR are formed in the distal end side portion 21Ga1 of the pipe 21Ga of FIG. 23. The proximal end side portion 21Ga2 is spirally cut to form the coil portion 24.

### Modification 10

In the first embodiment described above, the pipe 21 has the slit portion 23 and the coil portion 24, but a multi-lumen tube may be inserted into the portion corresponding to the coil portion 24 without the coil portion 24 being provided.

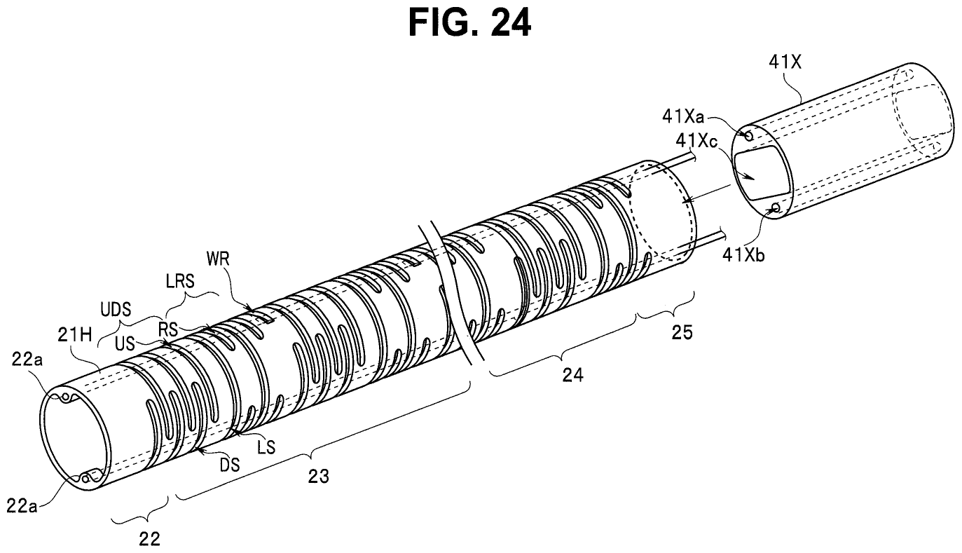

FIG. 24 is a configuration diagram of a bending portion 7 of the insertion portion according to a modification 10 of the first embodiment.

The bending portion 7 has a pipe 21H provided with a plurality of slit portions 23, and a multi-lumen tube 41X having an outer diameter that can fit into the inner diameter of the pipe 21H. The pipe 21H has a distal end connecting portion 22 on the distal end side, and a plurality of slit portions 23 are formed on the proximal end side of the distal end connecting portion 22 in the longitudinal direction of the pipe 21H. A wire receiving portion WR is provided between two adjacent slit portions 23.

A multi-lumen tube 41X is inserted into the proximal end portion of the pipe 21H and is fixed to the pipe 21H with an adhesive or the like. The multi-lumen tube 41H is made of an elastic member such as rubber.

The multi-lumen tube 41X has three conduits 41Xa, 41Xb, and 41Xc in the longitudinal axis direction. The two conduits 41Xa and 41Xb are arranged in up-down bending direction with the conduit 41Xc interposed between the two conduits. A bending wire 26 is inserted into each of the conduits 41Xa and 41Xb. Therefore, the inner diameter of each of the conduits 41Xa and 41Xb is a size that allows the bending wire 26 to move in the longitudinal direction. The conduit 41Xc has a size into which internal components such as a signal cable 14 and a light guide 15 can be inserted, and is formed along the central axis of the multi-lumen tube 41.

The portion into which the multi-lumen tube 41H is inserted exhibits the same function as the coil portion 24 of the first embodiment.

Such a configuration can also produce the same effect as the effect of the bending portion 7 of the first embodiment.

### Modification 11

In the first embodiment described above, the pipe 21 is covered with a resin tube, but the pipe 21 may be further covered with an outer tube.

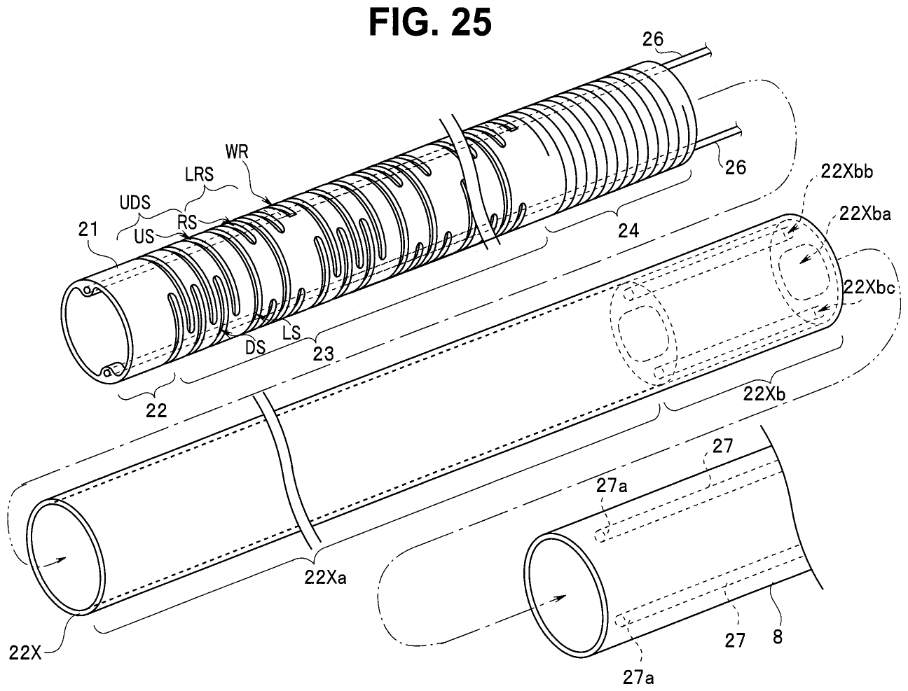

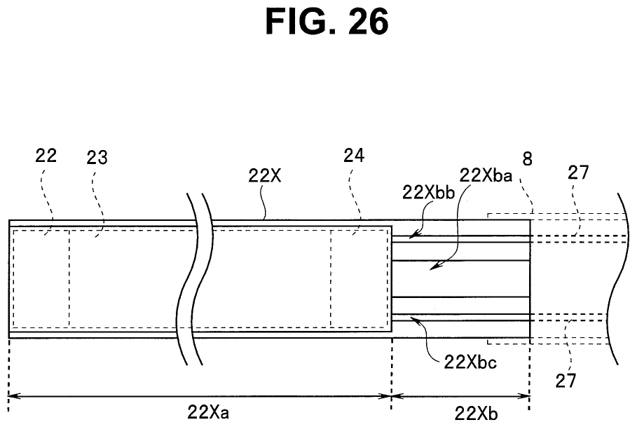

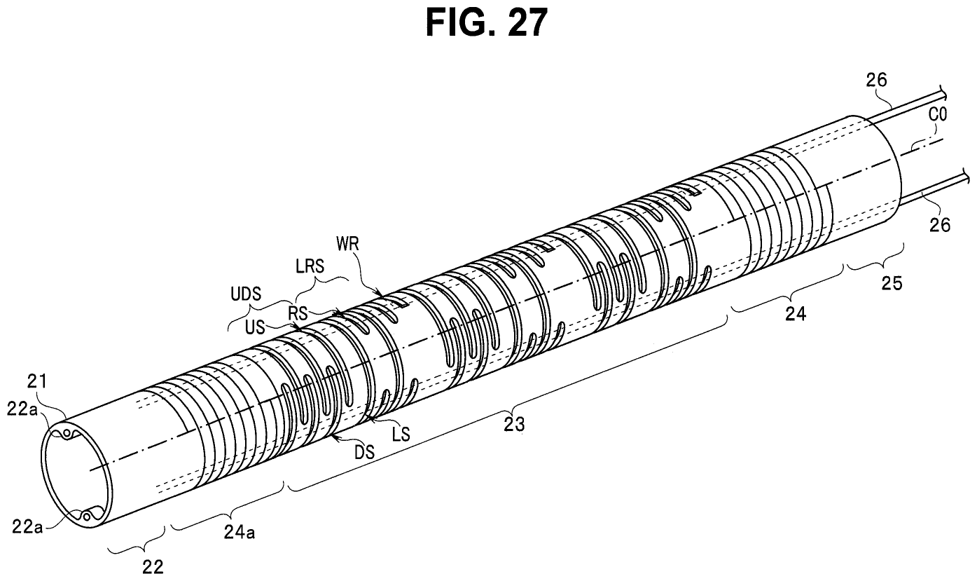

FIG. 25 is a configuration diagram of a bending portion 7 of the insertion portion according to a modification 11 of the first embodiment. FIG. 26 is a cross-sectional view of the bending portion 7 of the insertion portion according to a modification 11 of the first embodiment.

The bending portion 7 is inserted into an elastic tube 22X. More specifically, the tube 22X has a tubular portion 22Xa having an inner diameter into which the bending portion 7 can be inserted on the distal end side. The tube 22X has a solid portion 22Xb having three conduits 22Xba, 22Xbb, and 22Xbc on the proximal end side of the tubular portion 22Xa. The solid portion 22Xb configures a multi-lumen tube.

The proximal end portion of the solid portion 22Xb is inserted into the distal end side of the flexible tube portion 8. Two coil pipes 27 are inserted into the flexible tube portion 8. The bending portion 7 is inserted and arranged in the tubular portion 22Xa so that the two bending wires 26 are inserted into the two conduits 22Xbb and 22Xbc and the two coil pipes 27.

Such a configuration can cover the bending portion 7 instead of the resin tube, and produce the same effect as the effect of the bending portion 7 of the first embodiment.

### Modification 12

In the first embodiment described above, the pipe 21 has the distal end connecting portion 22 on the distal end side, but the pipe 21 may be provided with another coil portion on the distal end side of the pipe 21 between the distal end connecting portion 22 and the slit portion 23.

FIG. 27 is a configuration diagram of a bending portion 7 of the insertion portion according to a modification 12 of the first embodiment.

In the modification, a second coil portion 24a is provided on the distal end side of the slit portion 23 between the distal end connecting portion 22 and the slit portion 23. The coil portion 24a is also formed by cutting the pipe 21 into a spiral shape around the central axis C0 in the same manner as the coil portion 24.

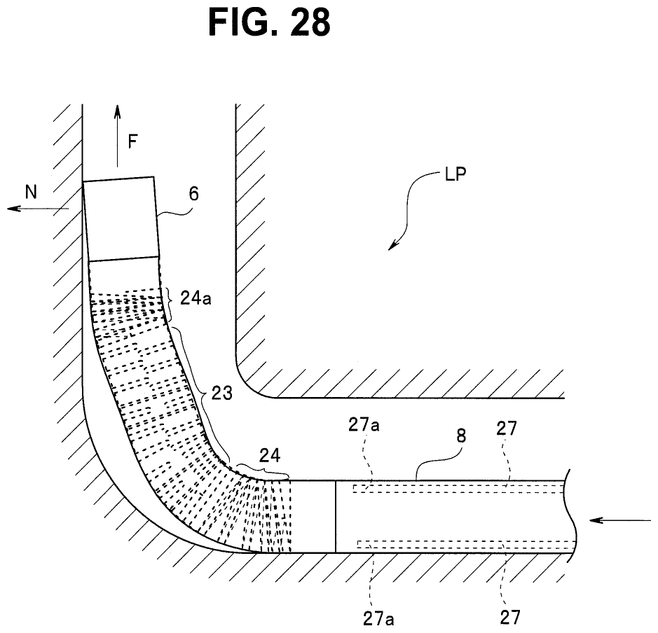

FIG. 28 is a diagram for explaining an action when the distal end portion of the insertion portion 4 of the endoscope having the structure of the modification 12 of the first embodiment is pushed into the L-shaped pipe LP. As shown in FIG. 28, when the distal end rigid portion 6 turns the elbow portion of the pipe LP and then further advances, the coil portion 24a at the distal end side portion of the bending portion 7 is deformed, which reduces the stress applied to one or more slits of the slit portion 23. In addition, the coil portion 24 on the proximal end side is also deformed, which further reduces the force of the distal end rigid portion 6 that pushes the inner wall of the pipe LP. As a result, it is possible to prevent the slit portion 23 of the bending portion 7 from being damaged.

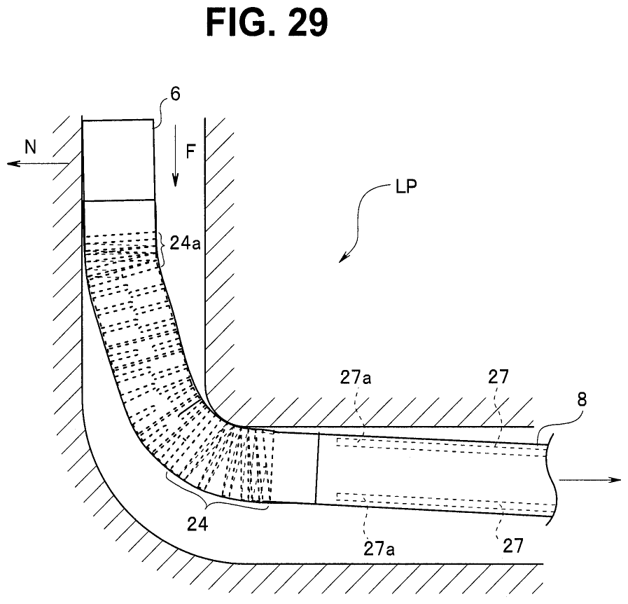

FIG. 29 is a diagram for explaining an action when the distal end portion of the insertion portion 4 of the endoscope having the structure of the modification 12 of the first embodiment is pulled out from the L-shaped pipe LP. As shown in FIG. 29, when the distal end rigid portion 6 approaches the elbow portion of the pipe LP and then is further pulled out, the coil portion 24 at the proximal end side portion of the bending portion 7 is greatly deformed, which reduces the stress applied to one or more slits of the slit portion 23. In addition, the coil portion 24a on the distal end side is also deformed, which further reduces the force of the distal end rigid portion 6 that pushes the inner wall of the pipe LP. As a result, it is possible to prevent the slit portion 23 of the bending portion 7 from being damaged.

Such a configuration can also produce the same effect as the effect of the bending portion 7 of the first embodiment.

As described above, according to the present embodiment and respective modifications described above, it is possible to provide an endoscope in which the bending portion 7 of the insertion portion 4 is hard to break.

### Second Embodiment

In the first embodiment, the bending portion 7 has a pipe 21 including a slit portion 23, but the bending portion of the present embodiment includes a coil member and a plurality of ring-shaped pipe members.

Since the endoscope system of the present embodiment has substantially the same configuration as the endoscope system 1 of the first embodiment, the description of the same components as the endoscope system 1 of the first embodiment is omitted here, and only the different components are described below.

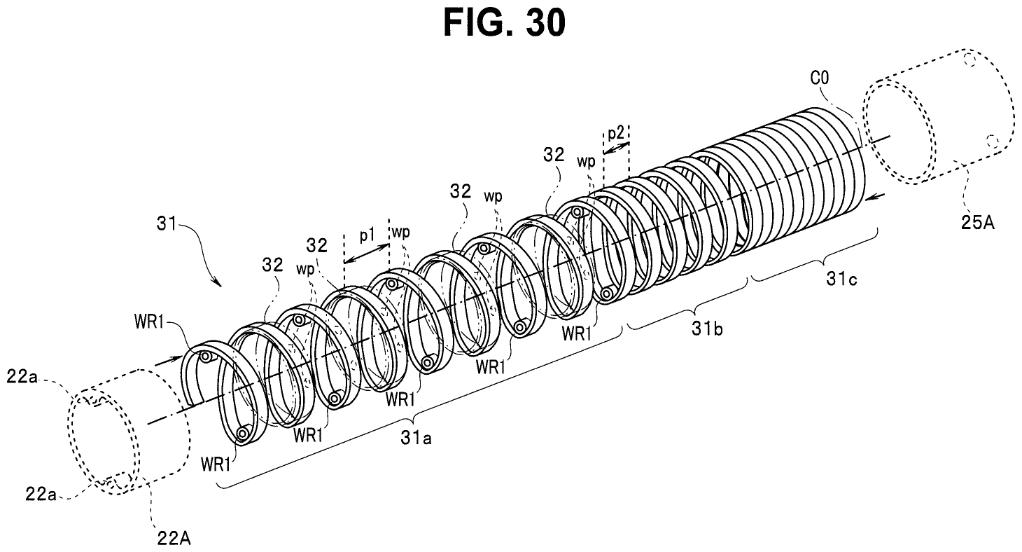

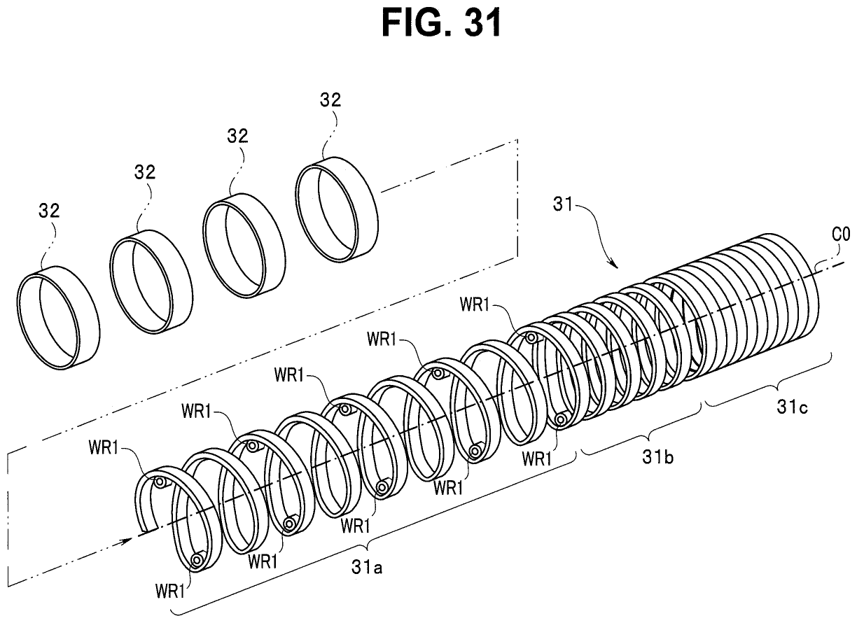

FIG. 30 is a perspective view of a coil used for a bending portion of the present embodiment. FIG. 31 is an assembly diagram of the bending portion of the present embodiment. As shown in FIG. 30, a coil 31 is made of a metal such as an alloy of nickel and titanium (Ni-Ti alloy), and is produced by cutting a pipe, which is a tubular member, into a spiral shape by laser processing.

The coil 31 is disposed in the bending portion 7 of the insertion portion 4 instead of the pipe 21 having the slit portion of the first embodiment. A distal end connecting portion 22A shown by the dotted line is fixed to the distal end side of the coil 31 by welding.

The coil 31 has a first coil portion 31a, a second coil portion 31b, and a third coil portion 31c in the order from the distal end side of the small-diameter insertion portion 4.

The first coil portion 31a is a coarsely wound coil portion. The second coil portion 31b is a coarsely wound coil portion having a winding pitch width p2 shorter than the pitch width p1 of the first coil portion 31a. The third coil portion 31c is a tightly wound coil portion formed so that the wound thin plate-shaped members are in close contact with each other. A plurality of pipe-shaped wire receiving portions WR1 are fixedly provided on the inner peripheral surface of the first coil portion 31a by welding or the like.

As shown in FIG. 30, a plurality of (four here) ring-shaped pipes 32 are disposed in the first coil portion 31a at predetermined intervals. The respective pipes 32 (indicated by a dashed and double-dotted line) are fixed to the inside of the first coil portion 31a by spot welding or laser welding at a plurality of positions (for example, three positions) wp (indicated by a dotted line).

The distal end connecting portion 22A is fitted into the recess portion 6a on the proximal end side of the distal end rigid portion 6 and fixed to the distal end rigid portion 6 with an adhesive or a screw (not shown).

The flexible tube portion connecting portion 25A shown by the dotted line is fixed to the proximal end portion of the coil 31 by welding or the like.

Note that laser processing may be applied to one pipe member to integrally form the distal end connecting portion 22A, the coil 31 and the flexible tube portion connecting portion 25A.

The distal end connecting portion 22A is provided with the two convex portions 22a for fixing the distal ends of the two bending wires 26.

As shown in FIG. 2, a bending wire 26 is inserted into the coil 31, two coil pipes 27 are fixed to the distal end portion of the flexible tube portion 8, and the insertion portion 4 can be bent in the up-down direction.

Since the plurality of pipes 32 are arranged and fixed in the first coil portion 31a at predetermined intervals, the gap between two adjacent pipes 32 shrinks or expands when the first coil portion 31a is bent, so that the first coil portion 31a can be bent.

As described above, there are disposed the first coil portion 31a, which is a coarsely wound coil, and the plurality of the pipes 32 fixed inside the first coil portion 31a in the region corresponding to the slit portion 23 of the first embodiment, and there is disposed a third coil portion 31c, which is a tightly wound coil, in the region corresponding to the coil portion 24 of the first embodiment. The plurality of (four here) ring-shaped pipes 32 are disposed in the first coil portion 31a at predetermined intervals, so that the first coil portion 31a is not crushed.

The function of the coil 31 of the present embodiment has the same function as the function of the pipe 21 of the first embodiment. In other words, when the user pushes the insertion portion 4 into the conduit that is the object, or pulls out the insertion portion 4 from the conduit, the third coil portion 31c is greatly deformed, which reduces the stress applied to the first coil portion 31a and the second coil portion 31b. As a result, it is possible to prevent the first coil portion 31a and the second coil portion 31b of the bending portion 7 from being damaged.

As described above, according to the present embodiment, it is possible to provide an endoscope apparatus in which the bending portion 7 of the insertion portion 4 is hard to break.

### Third Embodiment

In the first embodiment, the bending portion 7 is formed by processing the pipe 21, but a bending portion of the present embodiment is formed by processing a multi-lumen tube. In other words, the bending portion of the present embodiment includes a multi-lumen tube having a slit portion with a plurality of slits, and a coil portion.

Since the endoscope system of the present embodiment has substantially the same configuration as the endoscope system 1 of the first embodiment, the description of the same components as the endoscope system 1 of the first embodiment is omitted here, and only the different components are described below.

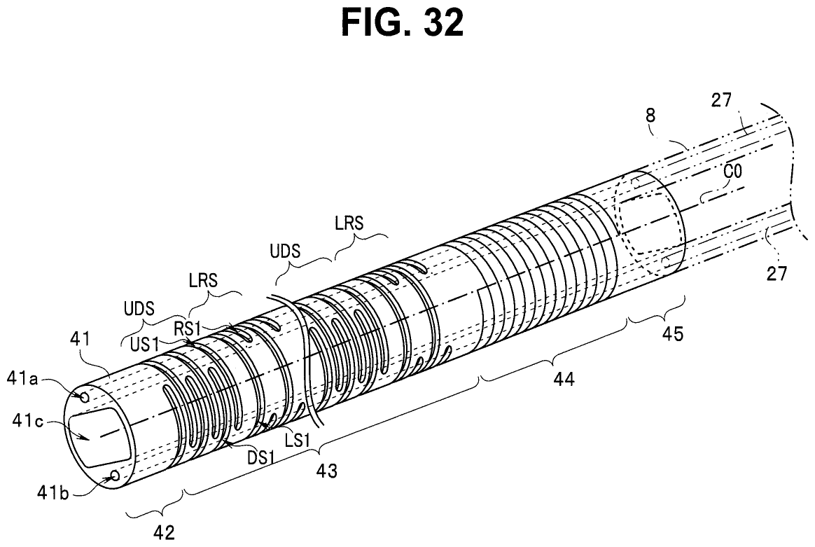

FIG. 32 is a perspective view of the multi-lumen tube used for the bending portion of the present embodiment. As shown in FIG. 32, the multi-lumen tube 41 is a tube having a circular cross-sectional shape and having a plurality of (three here) lumens (that is, conduits) formed in the longitudinal direction. The multi-lumen tube 41 is made of an elastic metal such as a nickel titanium alloy (Ni-Ti alloy).

The multi-lumen tube 41 is disposed in the bending portion 7 of the small-diameter insertion portion 4 instead of the pipe 21 of the first embodiment. The multi-lumen tube 41 includes a distal end connecting portion 42, a slit portion 43, a coil portion 44, and a flexible tube portion connecting portion 45 in the order from the distal end side. The slit portion 43 and the coil portion 44 are formed by laser processing. The distal end side portion of the distal end connecting portion 42 is fitted and fixed into the recess portion 6a (FIG. 2) formed on the proximal end side of the distal end rigid portion 6. The outer peripheral portion of the multi-lumen tube 41 fixed to the distal end rigid portion 6 is covered with the resin tube 7a (FIG. 2).

The multi-lumen tube 41 includes three conduits 41a, 41b, and 41c in the longitudinal axis direction. The two conduits 41a and 41b are arranged in up-down bending direction with the conduit 41c interposed between the two conduits. A bending wire 26 is inserted into each of the conduits 41a and 41b. Therefore, the inner diameter of each of the conduits 41a and 41b has a size that allows the bending wire 26 to move in the longitudinal direction. The distal end of each bending wire 26 is fixed to the multi-lumen tube 41 at the distal end portion of each of the conduits 41a and 41b by welding or an adhesive.

The conduit 41c has a size into which internal components such as a signal cable 14 and a light guide 15 can be inserted, and is formed along the central axis of the multi-lumen tube 41.

Similarly to the slit portion 23 of the first embodiment, the slit portion 43 has a plurality of slits or a plurality of cuts formed in a direction orthogonal to the longitudinal axis of the multi-lumen tube 41. The slit portion 43 includes: an upper and lower slit portion UDS in which a plurality of (six here) slits are formed on the upper side and the lower side of the multi-lumen tube 41 so as to bend in the up-down direction; and a left and right slit portion LRS in which a plurality of (four here) slits are formed on the right and left sides of the multi-lumen tube 41.

In the upper and lower slit portion UDS, there are alternately formed: the upper side slits US1 each being a slit formed on the upper side (that is, the upper bending direction side) of the multi-lumen tube 41; and the lower side slits DS1 each being a slit formed on the lower side (that is, the lower bending direction side) of the pipe 21 of the multi-lumen tube 41.

In the left and right slit portion LRS, there are alternately formed: the right side slits RS1 each being a slit formed on the right side (that is, the right bending direction side) of the multi-lumen tube 41; and the left side slits LS1 each being a slit formed on the left side (that is, the left bending direction side) of the multi-lumen tube 41.

The flexible tube portion 8 is formed of, for example, a multi-lumen tube made of resin, and has a shape to which a bending portion 7 made of a multi-lumen tube 41 is connected at the distal end. The flexible tube portion 8 has a shape substantially identical to the shape of the bending portion 7, and has a conduit 27 for arranging the bending wire 26.

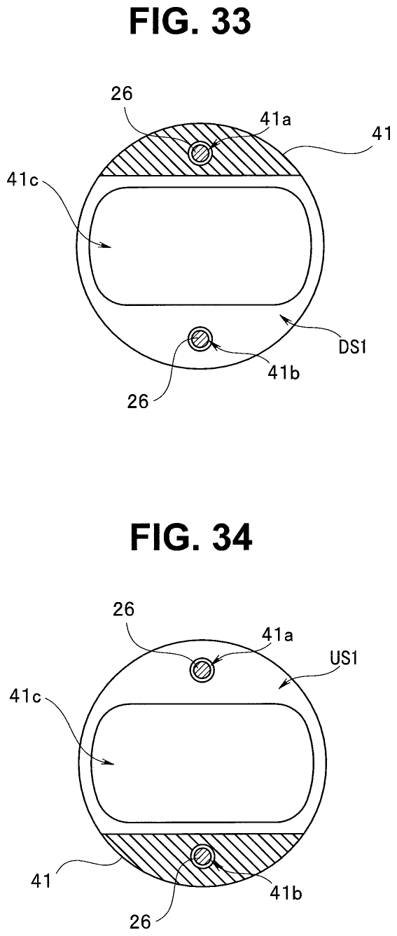

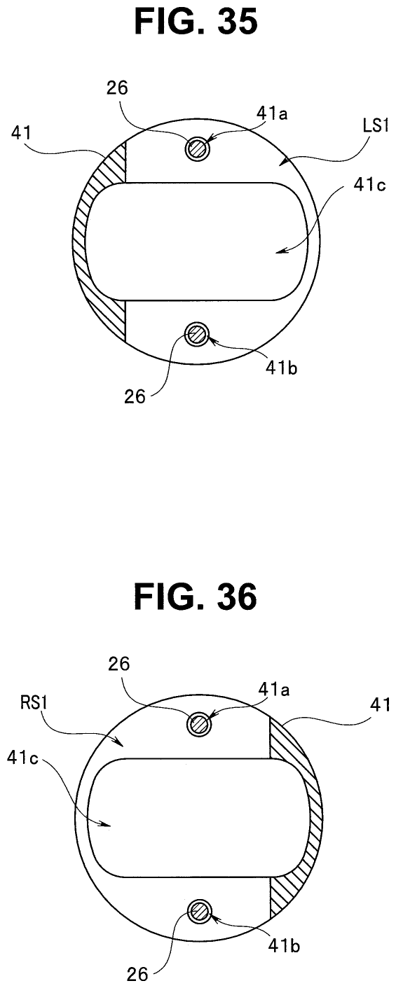

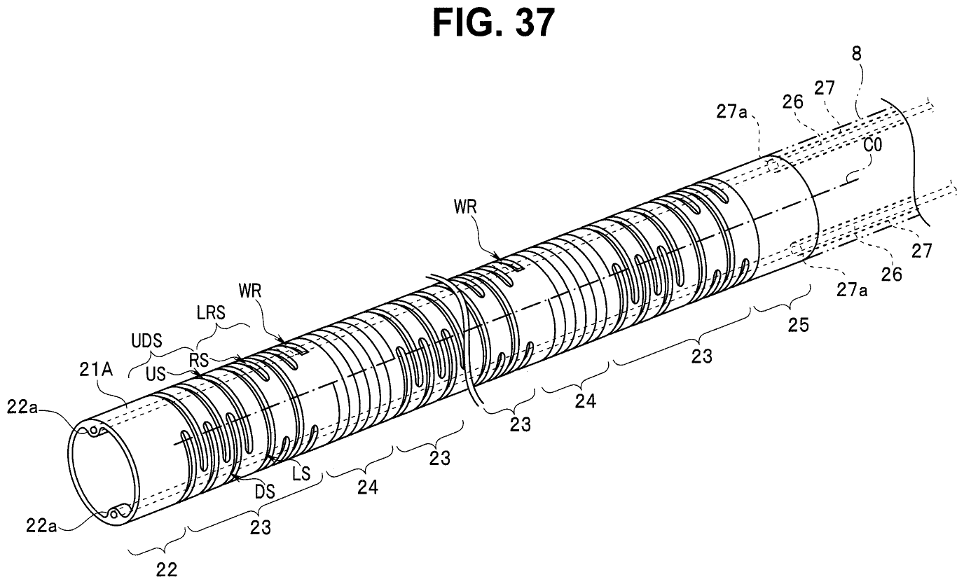

FIG. 33 is a cross-sectional view of a portion where the lower side slit DS1 of the multi-lumen tube 41 is formed when the multi-lumen tube 41 is viewed from the distal end side. FIG. 34 is a cross-sectional view of a portion where the upper side slit US1 of the multi-lumen tube 41 is formed when the multi-lumen tube 41 is viewed from the distal end side. FIG. 35 is a cross-sectional view of a portion where the left side slit LS1 of the multi-lumen tube 41 is formed when the multi-lumen tube 41 is viewed from the distal end side. FIG. 36 is a cross-sectional view of a portion where the right side slit RS1 of the multi-lumen tube 41 is formed when the multi-lumen tube 41 is viewed from the distal end side.

The coil portion 44 is formed such that the coil portion 44 is spirally cut around the central axis C0 so as to make a cut from the outer surface of the multi-lumen tube 41 to the space formed by the conduit 41c.

Note that the portion of the coil portion 44 may have a normal coil shape instead of a general multi-lumen shape. In this case, the portion of the coil portion 44 can be processed by processing the inner surface from one side of the tube-shaped multi-lumen 41 formed into a multi-lumen shape in advance to provide a portion having a normal pipe shape. Furthermore, there may be a structure such that only a part of the coil portion 44 is of non multi-lumen shape, instead of the entire coil portion 44. The portion of the coil portion 41 is provided with a portion having a larger space than the bending portion 7 on the distal end side and the flexible tube portion 8 on the proximal end side, so that a margin is provided for the internal component, which allows the internal component to easily slide for the bending operation and the deformation of the flexible tube portion 8, and allows easy bending operation.

As described above, the slit portion 43 and the coil portion 44 are formed by processing the multi-lumen tube 41. The function of the multi-lumen tube 41 of the present embodiment has the same function as the function of the pipe 21 of the first embodiment. In other words, when the user pushes the insertion portion 4 into the conduit which is the object, or when the user pulls out the insertion portion 4 from the inside of the conduit, a great deformation on the coil portion 44 reduces the stress applied to the slit portion 43. As a result, damage to the slit portion 43 can be prevented.

Note that, in the above-mentioned example, the multi-lumen tube 41 is made of metal, but may be made of resin. In the case, the multi-lumen tube 41 made of resin may be extended so as to be a part of the flexible tube portion 8 as shown by the dashed and double-dotted line in FIG. 30.

As described above, according to the present embodiment, it is possible to provide an endoscope apparatus in which the bending portion of the insertion portion is hard to break.

### Fourth Embodiment

In the first embodiment, the bending portion has one slit portion and one coil portion arranged on the proximal end side of the slit portion, but the bending portion of the present embodiment includes a plurality of slit portions and coil portions each between two of the plurality of slit portions adjacent to each other.

Since the endoscope system of the present embodiment has substantially the same configuration as the endoscope system 1 of the first embodiment, the description of the same components as the endoscope system 1 of the first embodiment is omitted here, and only the different components are described below.

FIG. 37 is a configuration diagram of a bending portion 7 of a small-diameter insertion portion according to the present embodiment. The pipe 21A has a distal end connecting portion 22 on the distal end side, and a flexible tube portion connecting portion 25 on the proximal end side. Between the distal end connecting portion 22 and the flexible tube portion connecting portion 25, there are provided a plurality of slit portions 23 and a plurality of coil portions 24 provided between two of the slit portions 23 adjacent to each other. In other words, a plurality of slit portions 23 and a plurality of coil portions 24 are provided so as to be alternately arranged. The slit portion 23 and the coil portion 24 are formed by laser processing.

The plurality of the slit portions 23 and the plurality of the coil portions 24 may be formed by processing one pipe 21A by laser processing, or the slit portions and the coil portions 24 manufactured separately may be connected by welding or the like to integrally form the plurality of slit portions 23 and the plurality of coil portions 24.

The slit portion 23 includes the upper and lower slit portions UDS and the left and right slit portions LRS, similarly to the first embodiment. Wire receiving portions WR are formed at a plurality of positions between the slit portions 23 and the coil portions 24. The wire receiving portion WR is a space formed inside the pipe 21 by lance bending processing.

Note that a slit portion 23 is disposed on the distal end side of the flexible tube portion connecting portion 25 in FIG. 37, but a coil portion 24 may be disposed.

The coil portion 24 is provided between two of the slit portions 23 adjacent to each other, so that the coil portions 24 at a plurality of positions of the bending portion 7 are bent to absorb a part of the stress applied to the slit portions 23 on the distal end side, which can reduce the stress applied to the bending portion 7.

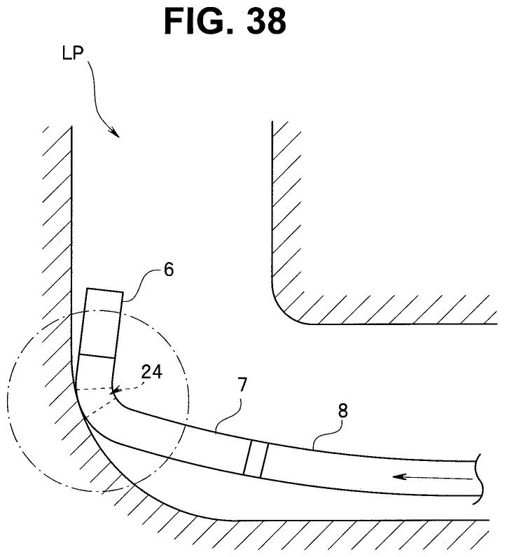

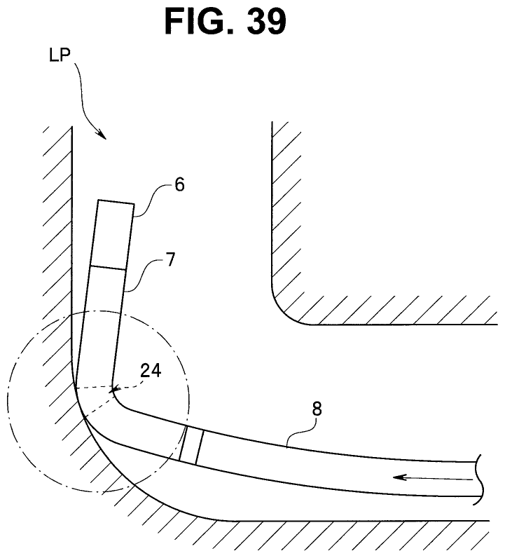

FIG. 38 is a diagram for explaining an action when the distal end portion of the insertion portion 4 of the endoscope having the structure of the present embodiment is pushed into the L-shaped pipe LP. As shown in FIG. 38, when the distal end rigid portion 6 turns the elbow portion of the pipe LP and then further advances, one coil portion 24 (indicated by the dotted line) disposed near the distal end side portion of the bending portion 7 is greatly deformed, which reduces the stress applied to one or more slits of the slit portion 23 located on the distal end side of the coil portion 24.

FIG. 39 is a diagram for explaining an action when the distal end portion of the insertion portion 4 of the endoscope having the structure of the present embodiment is pushed into the L-shaped pipe LP. As shown in FIG. 39, when the distal end rigid portion 6 turns the elbow portion of the pipe LP and then further advances, a load may be applied from the pipe LP to the middle portion of the bending portion 7. In such a case, one coil portion 24 (indicated by a dotted line) disposed in the middle portion of the bending portion 7 is greatly deformed, which reduces the stress applied to one or more slits of the slit portion 23 located on the distal end side of the coil portion 24.

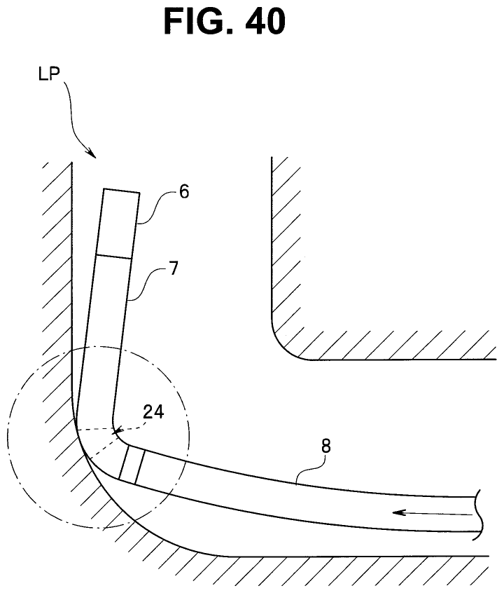

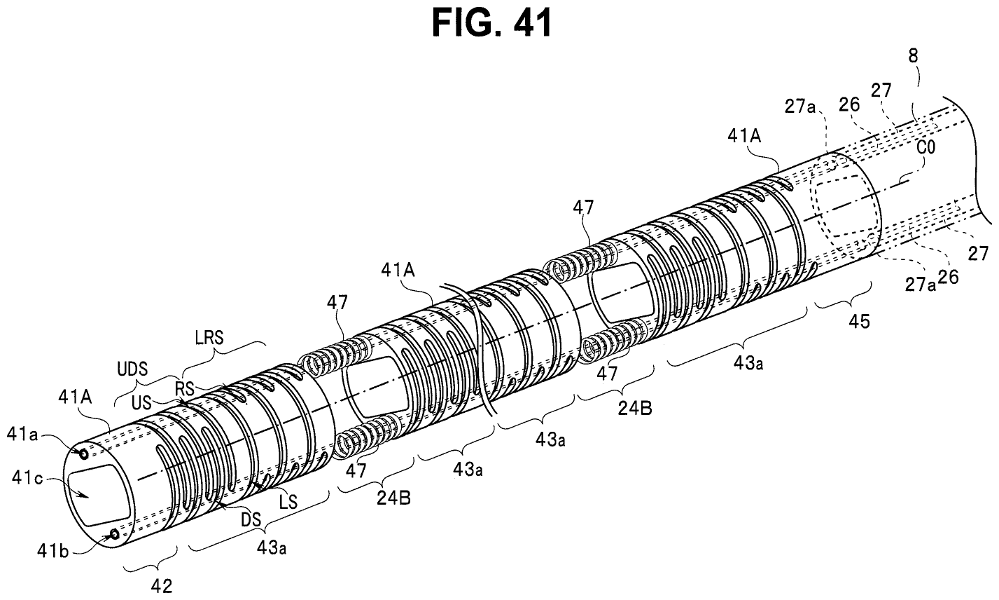

FIG. 40 is a diagram for explaining an action when the distal end portion of the insertion portion 4 of the endoscope having the structure of the present embodiment is pushed into the L-shaped pipe LP. As shown in FIG. 40, when the distal end rigid portion 6 turns the elbow portion of the pipe LP and then further advances, a load may be applied from the pipe LP to the proximal end side portion of the bending portion 7. In such a case, one coil portion 24 (indicated by the dotted line) disposed in the vicinity of the proximal end side portion of the bending portion 7 is greatly deformed, which reduces the stress applied to one or more slits of the slit portion 23 located on the distal end side of the coil portion 24.

When the bending portion 7 moves with respect to the elbow portion of the pipe LP, the position of the bending portion 7 in contact with the pipe LP may change little by little, but no matter where the bending portion 7 is bent, the stress on the slit is relatively relieved.

In the cases described with reference to FIGS. 38 to 40, the same effect is obtained when the insertion portion 4 is pulled out when the inner diameter of the pipe LP is small.

### Modification 4-1

In the fourth embodiment described above, each coil portion 24 disposed between two of the slit portions 23 adjacent to each other has the same diameter as the slit portion 23, but may be configured with a plurality of coils each having a diameter different from the diameter of the slit portion 23.

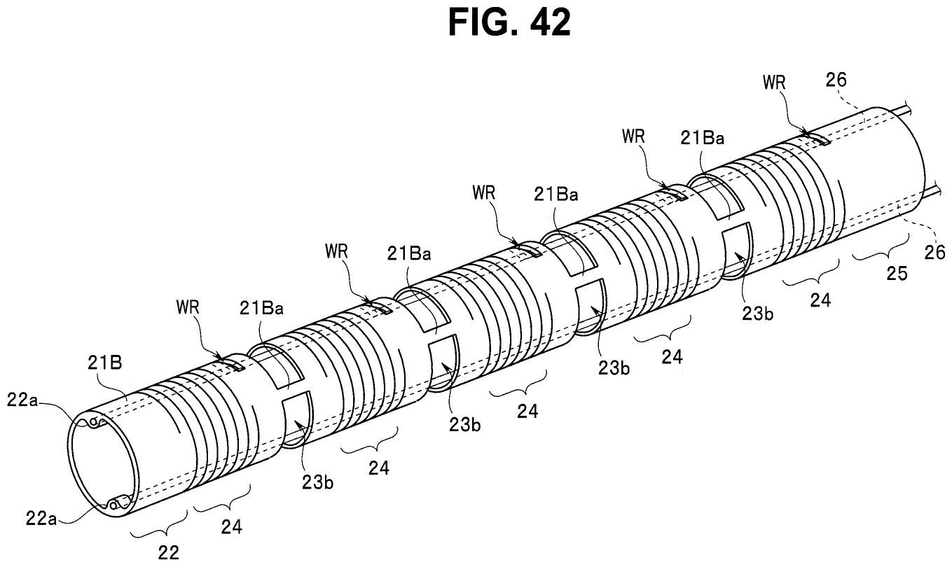

FIG. 41 is a configuration diagram of a bending portion 7 of the insertion portion according to a modification 4-1 of the fourth embodiment. Respective slit portions of the modification configure slit portions 43a each having the form of the multi-lumen tube 41 described in the third embodiment. A coil portion 24B is provided between two of the slit portions 43a adjacent to each other.

As shown in FIG. 41, a distal end connecting portion 42 is formed on the distal end side of a slit portion 43a arranged on the distalmost end side of the bending portion 7. A flexible tube portion connecting portion 45 is formed on the proximal end side of the slit portion 43a arranged on the proximalmost end side of the bending portion 7.

The multi-lumen tube 41A has three conduits 41a, 41b, and 41Ac in the longitudinal axis direction. The two conduits 41a and 41b are arranged in up-down bending direction with the conduit 41c interposed between the two conduits. A bending wire 26 is inserted into each of the conduits 41a and 41b. Therefore, the inner diameter of each of the conduits 41a and 41b has a size that allows the bending wire 26 to move in the longitudinal direction. The distal ends of respective bending wire 26 are fixed to the multi-lumen tube 41A by welding or an adhesive at the distal end portions of the conduits 41a and 41b of the distal end connecting portion 42 arranged at the distalmost.

Each coil portion 24B is configured with two coils 47. The inner diameter of each coil 47 has a size into which one bending wire 26 can be inserted. The two coils 47 are fixed to the two slit portions 43a by welding or the like, so as to connect the openings of the conduit 41a and 41b of the slit portion 43a on the distal end side of two slit portions 43a adjacent to each other to the openings of the conduits 41a and 41b of the slit portion 43a on the proximal end side of the two slit portions 43a adjacent to each other.

Also in the modification, each coil portion 24B absorbs a part of the stress applied to the slit portion 43 connected to the distal end side.

### Modification 4-2

In the modification 4-1 of the fourth embodiment described above, a plurality of coils each having a diameter different from the diameter of the slit portion 23 are provided between two slit portions 23 adjacent to each other, but each slit portion may be configured such that the pipe is cut out to leave only a part of the pipe.

FIG. 42 is a configuration diagram of a bending portion 7 of the insertion portion according to a modification 4-2 of the fourth embodiment.

A coil portion 24 is formed by spirally cutting around the central axis C0 from a position separated from the distal end side by a predetermined distance, so as to form the distal end connecting portion 22 on the distal end side of the pipe 21B. The pipe 21B is cut so that a slit portions 23b is formed with two connecting portions 21Ba being left at the proximal end portion of the coil portion 24. The pipe 21B is processed so that two connecting portions 21Ba are formed on a line that passes through the central axis C0 and is parallel to the left-right direction when the bending portion 7 is viewed from the distal end side. The processing for forming the coil portions 24 and the slit portions 23b on the pipe 21B is performed by, for example, laser processing.

One coil portion 24 and one slit portion 23b are formed on the proximal end side of each slit portion 23b. Then, as shown in FIG. 42, a plurality of slit portions 23b and a plurality of coil portions 24 are formed in the same manner. Note that wire receiving portions WR into which the bending wire 26 is inserted are formed in the vicinity of the coil portion 24.

Also in the modification, respective coil portions 24 absorb a part of the stress applied to the slit portion 23b connected to the distal end side.

### Modification 4-3

In the modification 4-2 of the fourth embodiment described above, a slit portion 23b is provided, between two of the coil portions 24 adjacent to each other, such that the pipe is cut out so as to leave only a part of the pipe, but a slit portion may be configured such that a plurality of pipes are used, and two adjacent pipe members fit each other at two points so as to be rotatable around a predetermined axis.

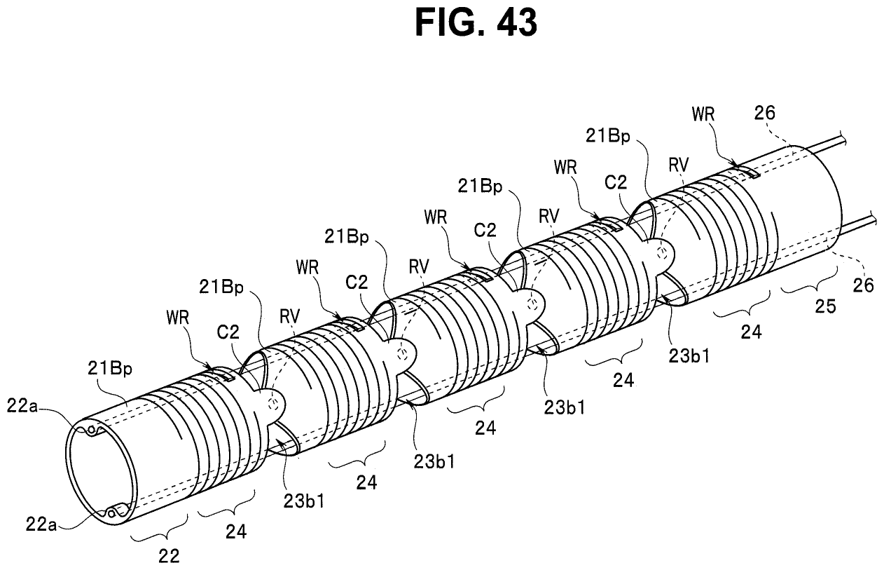

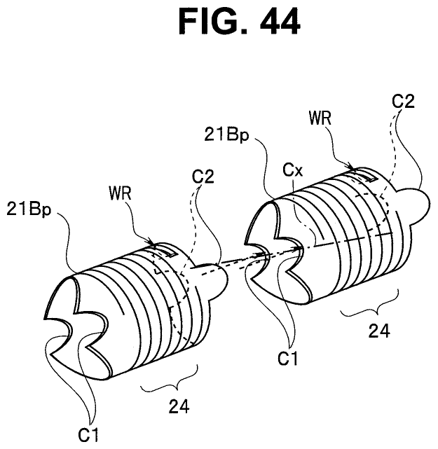

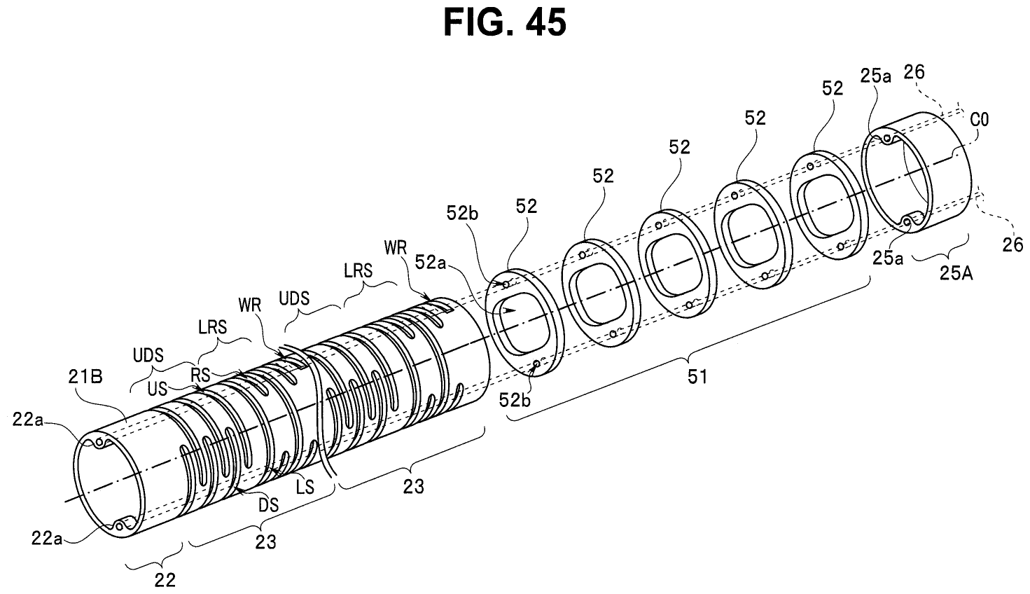

FIG. 43 is a configuration diagram showing the configuration of the bending portion 7 of the insertion portion according to a modification 4-3 of the fourth embodiment. FIG. 44 is a diagram for explaining the fitting of two of the pipes adjacent to each other according to the modification 4-3 of the fourth embodiment.

The bending portion 7 is configured with a plurality of pipes 21Bp being connected in series. A distal end connecting portion 22 is provided on the distal end side of the distalmost pipe 21Bp. A flexible tube portion connecting portion 25 is provided on the proximal end side of the proximalmost pipe 21Bp.

Concave portions C1 are formed at two positions on the distal end side of each pipe 21Bp excluding the distalmost pipe 21Bp, and convex portions C2 are formed at two positions on the proximal end side of each pipe 21Bp excluding the proximalmost pipe 21Bp. The tip shape of each convex portion C2 is a semicircular shape, and the shape of each concave portion C1 is a semicircular shape into which the convex portion C2 fits.

When the bending portion 7 is viewed from the distal end side, the two concave portions C1 are formed on an axis that passes through the central axis C0 and is parallel to the left-right direction. Similarly, the two convex portions C2 are also formed on an axis that passes through the central axis C0 and is parallel to the left-right direction when the bending portion 7 is viewed from the distal end side.

The concave portion C1 and the convex portion C2 that fit each other are rotatably connected around the axis passing through the center of the semicircle. As shown in FIG. 44, the two convex portions C2 on the proximal end side of one pipe 21Bp fit into the two concave portions C1 on the distal end side of another pipe 21Bp.

The two concave portions C1 and the two convex portions C2 fit each other, so that one pipe 21Bp is rotatable around a predetermined axis Cx with respect to the other pipe 21Bp. Furthermore, the slit portion 23b1 is formed by the gap formed around the fitting portion of the two concave portions C1 and the two convex portions C2.

Note that, here, the configuration of fitting the two concave portions C1 into the two convex portions C2 makes one pipe 21Bp rotatable around a predetermined axis Cx with respect to another pipe 21Bp, but a configuration of providing a pair of convex portions on both the distal end side and the proximal end side of each pipe 21Bp and connecting one pair of the convex portions to the other pair of convex portions by a rivet RV, as shown by the dotted line in FIG. 43, may be employed to make one pipe 21Bp rotatable around a predetermined axis Cx with respect to another pipe 21Bp.

Therefore, also in the modification, each coil portion 24 absorbs a part of the stress applied to the slit portion 23b1 connected to the distal end side.

As described above, according to the present embodiments and respective modifications, it is possible to provide an endoscope apparatus in which the bending portion 7 of the insertion portion 4 is hard to break.

As a related art, the above-described Japanese Patent Application Laid-Open Publication No. 2015-119839 proposes an endoscope having a structure in which a tubular bending portion is provided on the proximal end side of the active bending portion of the insertion portion to reduce the load applied to the active bending portion.

However, in the endoscope related to the proposal, the connection portion between the active bending portion and the tubular bending portion is a hard portion, and the distal end of the coil pipe for the bending wire is fixed on the distal end side of the tubular bending portion, so that a plurality of bending pieces in the proximal end portion of the active bending portion are damaged when a great load is applied to the proximal end portion of the active bending portion. In addition, if the active bending portion is configured such that a plurality of slits formed in the direction orthogonal to the longitudinal axis direction are provided in the longitudinal axis direction, the slits would rupture.

On the other hand, according to the above-described respective embodiments and the respective modifications, it is possible to provide an endoscope apparatus in which the bending portion of the insertion portion is hard to break.

Next, a disclosure example of another configuration of the bending portion of the endoscope is described below.

### First Disclosure Example

The bending portion of the present disclosure example is configured such that a plurality of plates is provided at the proximal end portion of the bending portion 7 instead of the coil portion 24.

Since the endoscope system of the present disclosure example has substantially the same configuration as the endoscope system 1 of the first embodiment, the description of the same components as the endoscope system 1 of the first embodiment is omitted here, and only the different components are described below.

FIG. 45 is a configuration diagram of a bending portion 7 of a small-diameter insertion portion according to the first disclosure example. The pipe 21B has a plurality of slit portions 23, but does not have a coil portion 24. The pipe 21B has a distal end connecting portion 22 on the distal end side.

A plate portion 51 is provided on the proximal end side of the pipe 21B. The plate portion 51 is configured with a plurality of plates 52. Each plate 52 is a disk-shaped metal plate member, and has a hole 52a and two holes 52b arranged so as to sandwich the hole 52a. The hole 52a has a size into which the internal components of the insertion portion 4 such as a signal cable 14 and a light guide 15 can be inserted, and is formed at a substantially central portion of the plate 52.

A plurality of plates 52 are stacked and disposed so as to be in close contact with the proximal end portion of the pipe 21B such that the internal components such as a signal cable 14 and a light guide 15 inserted into the hollow portion of the pipe 21B are inserted into a plurality of the holes 52a, the bending wire 26 inserted into the pipe 21B is inserted into the two holes 52b, and the central axis of each plate 52 coincides with the central axis C0 of the insertion portion 4.

A flexible tube portion connecting portion 25A, which is a metal pipe, is disposed on the proximal end side of the plate portion 51. The flexible tube portion 8 is fitted into the flexible tube portion connecting portion 25A and fixed by an adhesive. The flexible tube portion connecting portion 25A also has two convex portions 25a having holes into which the bending wire 26 can be inserted, similarly to the two convex portions 22a of the distal end connecting portion 22.

The flexible tube portion connecting portion 25A provided on the proximal end side of the plate portion 51 is a portion to which the distal end portion of the flexible tube portion 8 is connected. The distal end portion of the flexible tube portion 8 is fitted into the cylindrical flexible tube portion connecting portion 25A and fixed by an adhesive.

According to the configuration shown in FIG. 45, since the plurality of plates 52 of the plate portion 51 are not fixed to each other, when a load is applied to the slit portion 23, the plurality of plates 52 are separated from each other, which reduces the stress applied to the slit portion 23. As a result, it is possible to prevent the slit portion 23 of the bending portion 7 from being damaged.

As described above, according to the first disclosure example, it is possible to provide an endoscope apparatus in which the bending portion 7 of the insertion portion 4 is hard to break.

### Second Disclosure Example

The bending portion of the present disclosure example includes a multi-lumen tube and a coil portion.

Since the endoscope system of the present disclosure example has substantially the same configuration as the endoscope system 1 of the first embodiment, the description of the same components as the endoscope system 1 of the first embodiment is omitted here, and only the different components are described below.

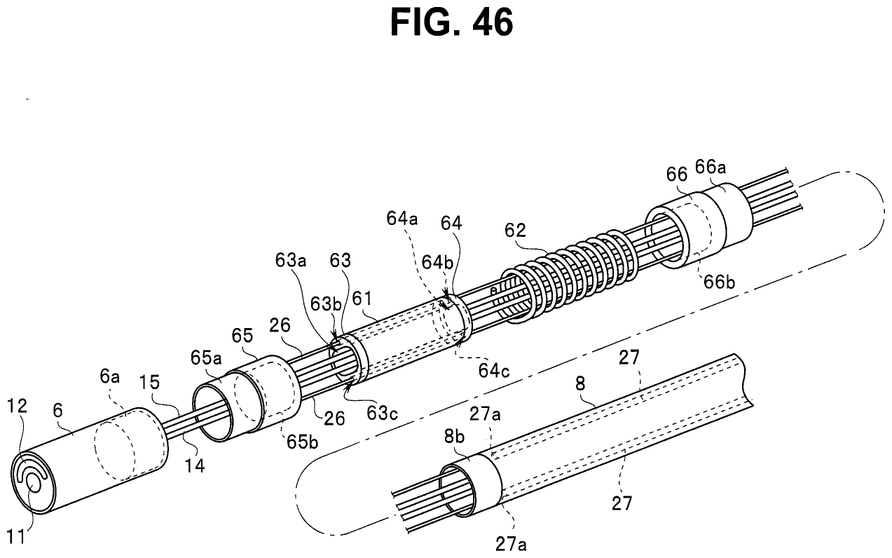

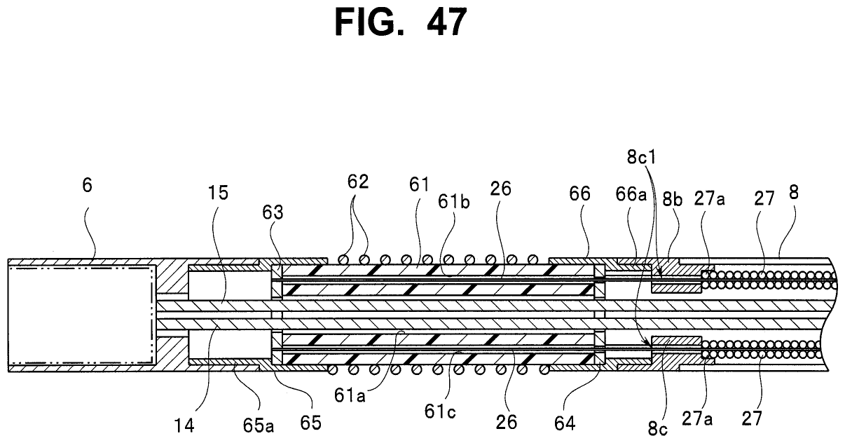

FIG. 46 is an assembly diagram of a bending portion 7 of a small-diameter insertion portion according to the second disclosure example. FIG. 47 is a cross-sectional view of the bending portion 7 in the longitudinal direction of the insertion portion 4 according to the second disclosure example.

The bending portion 7 includes a multi-lumen tube 61 and a coarsely wound coil 62. The multi-lumen tube 61 is inserted into the inner peripheral side of the coil 62. The multi-lumen tube 61 is made of a soft resin such as silicone. The multi-lumen tube 61 is formed with three holes 61a, 61b, and 61c. Of the three holes, the hole 61a along the central axis is a hole into which an internal component is inserted. The other two holes 61b and 61c of the three holes are holes into which the two bending wires 26 are inserted.

A disk-shaped front plate 63 and a rear plate 64 are arranged on the distal end side and the proximal end side of the multi-lumen tube 61, respectively. The front plate 63 is formed with three holes 63a, 63b, and 63c corresponding to the three holes 61a, 61b, and 61c of the multi-lumen tube 61. The rear plate 64 is also formed with three holes 64a, 64b, and 64c corresponding to the three holes 61a, 61b, and 61c of the multi-lumen tube 61.

A front pipe sleeve 65 is disposed on the distal end side of the multi-lumen tube 61. The front pipe sleeve 65 is a cylindrical metal member, and the front side has a stepped portion 65a having a small outer diameter. A recess portion 65b is formed inside the proximal end side portion of the front pipe sleeve 65. The distal end portion of the multi-lumen tube 61 is fitted into the recess portion 65b together with the front plate 63 and fixed by an adhesive.

The stepped portion 65a of the front pipe sleeve 65 is fitted into the recess portion 6a on the proximal end side of the distal end rigid portion 6 and fixed to the distal end rigid portion 6 with an adhesive or a screw (not shown).

A rear pipe sleeve 66 is disposed on the proximal end side of the multi-lumen tube 61. The rear pipe sleeve 66 is a cylindrical metal member, and the rear side has a stepped portion 66a having a small outer diameter. A recess portion 66b is formed inside the distal end side portion of the rear pipe sleeve 66. The proximal end portion of the multi-lumen tube 61 is fitted into the recess portion 66b together with the rear plate 64 and fixed by an adhesive.

The rear pipe sleeve 66 is a portion to which a pipe sleeve 8b of the distal end portion of the flexible tube portion 8 is connected. The cylindrical rear pipe sleeve 66 is fitted into the pipe sleeve 8b at the distal end portion of the flexible tube portion 8 and fixed by an adhesive. In addition, convex portions 8c on the inner peripheral surface of the pipe-shaped pipe sleeve 8b each have a conduit 8c1 into which the bending wire 26 is inserted, and the distal end portions 27a of the two coil pipes 27 are fixed to the end faces of the convex portions 8c with an adhesive or the like. Each bending wire 26 is inserted into each coil pipe 27.

Note that the circumference of the coil 62 covering the multi-lumen tube 61 configuring the bending portion 7 is covered with the resin tube 7a, which is not shown. The distal end side portion and the proximal end side portion of the resin tube 7a are thread-wound (not shown), coated with an adhesive, and fixed to the front pipe sleeve 65 and the rear pipe sleeve 66.

Furthermore, the flexible tube portion 8 includes a flexible tube into which an internal component such as a signal cable 14 is inserted, a braid covering the flexible tube, and a tubular outer skin covering the braid.

In addition, FIG. 48 is a cross-sectional view of the bending portion 7 orthogonal to the longitudinal direction of the insertion portion 4. The hole 61a is formed so as to include the central axis CC1 in the longitudinal direction of the multi-lumen tube 61. When the multi-lumen tube 61 is viewed from the distal end side, the two holes 61b and 61c into which the two bending wires 26 are inserted are disposed at a position deviated by a predetermined distance L from a plane CL1 that passes through the central axis CC1 and extends in up-down bending direction of the bending portion 7, so as to be plane symmetric with respect to a plane CL2 that is parallel to up-down bending direction, passes through the central axis CC1, and is perpendicular to the surface CL1.

Since the light guide 15 has a higher rigidity and is harder to bend than the signal cable 14, the two holes 61b and 61c are formed so as to be offset to the side of the light guide 15, which is harder (that is, harder to bend) than the signal cable 14, by a predetermined distance L from the central axis CC1, and so as to be plane symmetric with respect to plane CL2.

As a result, the bending portion 7 can be bent in the up-down direction, in bending the bending portion 7 in the up-down direction.

The predetermined distance L is determined by experiments or the like according to the hardness or softness of the multi-lumen tube 61, the light guide 15, and the signal cable 14.

Note that when the bending portion 7 is viewed from the distal end side, the shape of the holes 61a may be changed instead of changing the arrangement of the two holes 61b and 61c.

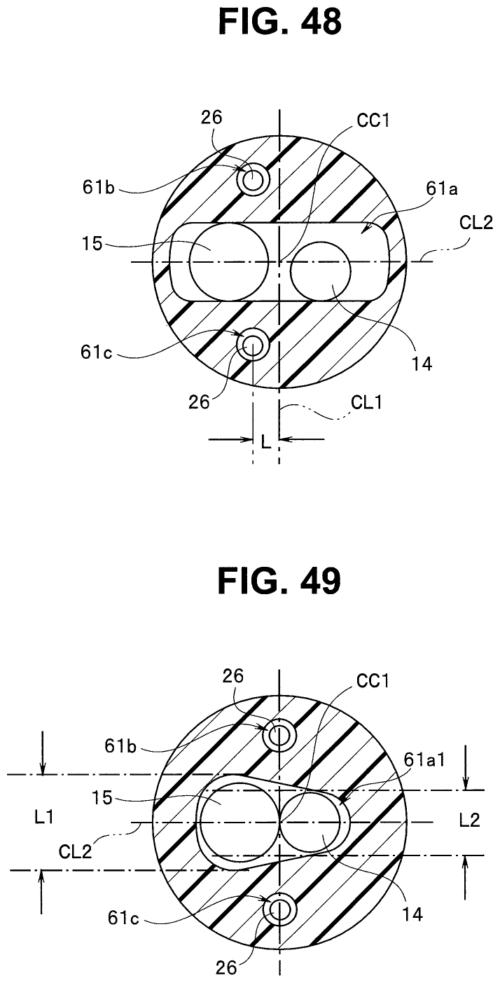

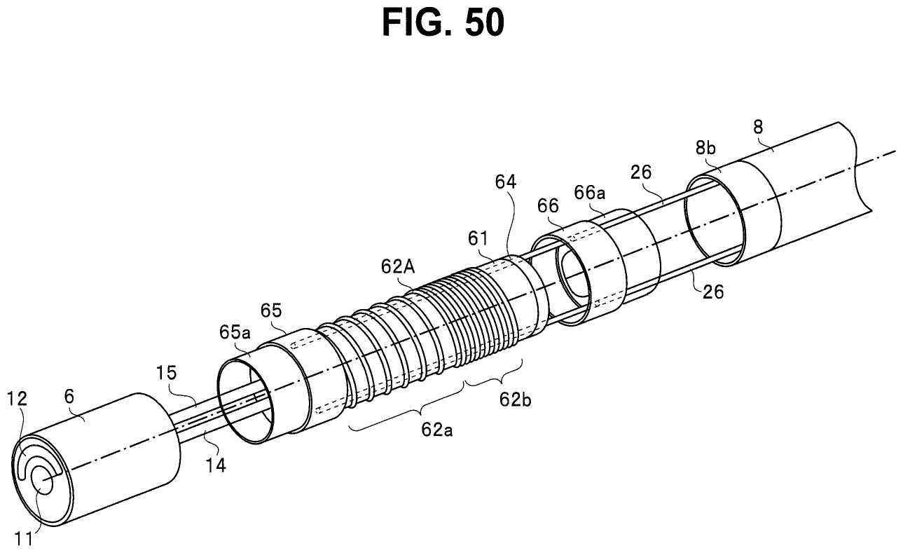

FIG. 49 is a cross-sectional view of the bending portion 7 orthogonal to the longitudinal direction of the insertion portion 4. The hole 61a1 is formed so as to include the central axis CC1 in the longitudinal direction of the multi-lumen tube 61, but the shape of the holes 61a1 when the multi-lumen tube 61 is viewed from the distal end side is not formed so as to be plane symmetric with respect to the plane CL1, that is, the shape of the holes 61a1 is formed so as to be non-plane symmetric.

The two holes 61b and 61c into which the two bending wires 26 are inserted are arranged so as to be formed in the surface CL1 and to be plane symmetric with respect to the surface CL2 when the multi-lumen tube 61 is viewed from the distal end side.

Since the light guide 15 has higher rigidity than the signal cable 14 and is hard to bend, the holes 61a has a reduced wall thickness of the multi-lumen tube 61 around the light guide 15 and an increased wall thickness of the multi-lumen tube 61 around the signal cable 14, so that the multi-lumen tube 61 around the light guide 15 is made easy to bend, and the multi-lumen tube 61 around the signal cable 14 is made hard to bend.

As shown in FIG. 49, in the direction parallel to the plane CL1, the width L1 in the up-down direction of the hole 61a1 of the multi-lumen tube 61 around the light guide 15 is greater than the width L2 in the up-down direction of the hole 61a1 of the multi-lumen tube 61 around the signal cable 14.

As a result, the bending portion 7 can be bent in the up-down direction, in bending the bending portion 7 in the up-down direction.

The widths L1 and L2 are determined by experiments and the like according to the hardness or softness of the multi-lumen tube 61, the light guide 15, and the signal cable 14.

According to the second disclosure example, it is possible to provide an endoscope apparatus in which the bending portion 7 of the insertion portion 4 is hard to break. Furthermore, according to the second disclosure example, since the coil 62 covers the circumference of the multi-lumen tube 61, the multi-lumen tube 61 is protected and is hard to break.

Note that the winding state of the coil 62 need not be uniform, and the distal end side may be coarsely wound and the proximal end side may be tightly wound.

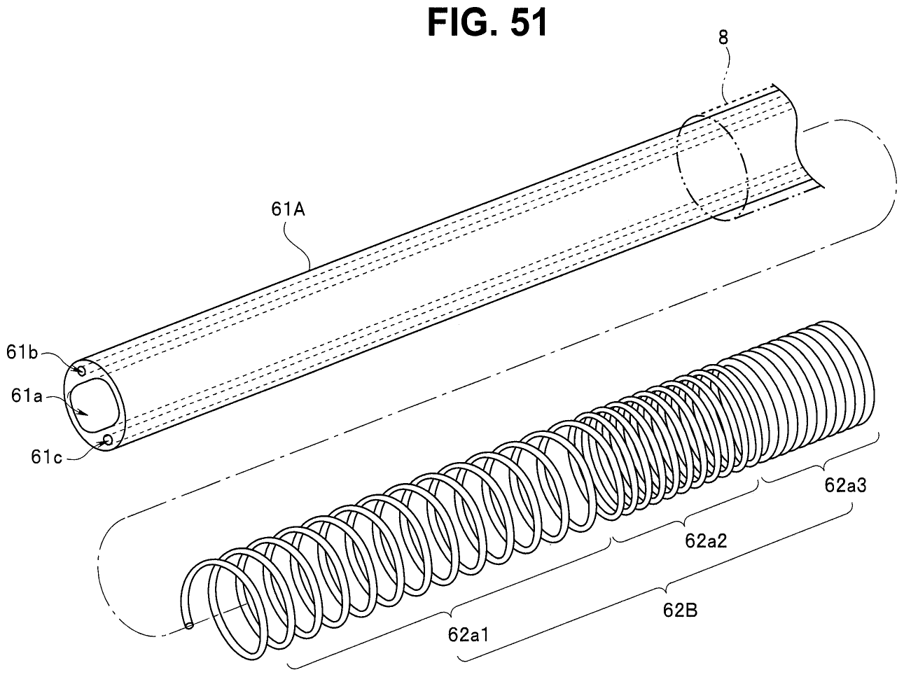

FIG. 50 is an assembly diagram showing a configuration of a bending portion 7 of an insertion portion using a coil in which the distal end side is coarsely wound and the proximal end side is tightly wound. In FIG. 50, the resin tube and the like covering the bending portion 7 are omitted.

The coil 62A has a coarsely wound portion 62a and a tightly wound portion 62b. The coil 62A is externally inserted into the multi-lumen tube 61 so that the distal end side is the coarsely wound portion 62a. According to such a configuration, the distal end side portion of the coil 62A of the bending portion 7 can be bent more easily than the proximal end side portion.

Furthermore, note that there may be a configuration such that: the multi-lumen tube of the bending portion 7 is lengthened so as to reach at least the distal end side portion of the flexible tube portion 8; and the distal end side of the coil covering the multi-lumen tube is coarsely wound, the proximal end side of the coil is tightly wound, and the intermediate portion between the distal end side portion and the proximal end side portion is a medium coarsely wound portion with a narrower winding pitch than the coarsely wound portion on the distal end side.

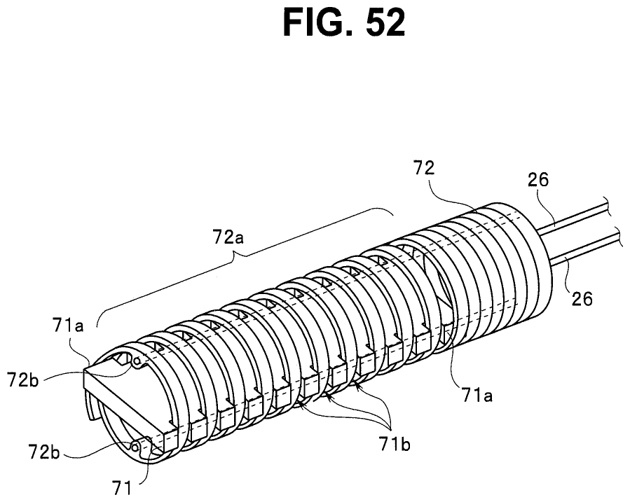

FIG. 51 is an assembly diagram showing a configuration of a bending portion 7 of an insertion portion in which a multi-lumen tube 61A is lengthened and an intermediate portion is provided between the coarsely wound portion and the tightly wound portion of the coil 62B. FIG. 51 omits the bending wire 26, the distal end rigid portion 6, and the like, and shows only the multi-lumen tube 61A and the coil 62B.

The elongated multi-lumen tube 61A has a length that reaches not only the bending portion 7 but also the inside of the flexible tube portion 8 on the proximal end side of the bending portion 7. The multi-lumen tube 61A is entirely disposed in the longitudinal direction of the flexible tube portion 8. The coil 62B covering the multi-lumen tube 61A is disposed in the bending portion 7. The coil 62B has a coarsely wound portion 62a1 on the distal end side and a tightly wound portion 62a3 on the proximal end side. The coil 62B has an intermediate portion 62a2 between the coarsely wound portion 62a1 and the tightly wound portion 62a3, which is not a tightly wound portion but a coarsely wound portion having a pitch shorter than the pitch of the coil of the coarsely wound portion 62a1.

According to such a configuration, the bending portion 7 is hard to break and the multi-lumen tube 61A extends to the flexible tube portion 8, so that less load is applied to the connection portion between the bending portion 7 and the flexible tube portion 8 to improve the insertability of the insertion portion 4 when the insertion portion 4 is pushed to be inserted into the bent object.

### Third Disclosure Example

The bending portion of the present disclosure example includes a plate-shaped member and a coil.

Since the endoscope system of the present disclosure example has substantially the same configuration as the endoscope system 1 of the first embodiment, the description of the same components as the endoscope system 1 of the first embodiment is omitted here, and only the different components are described below.

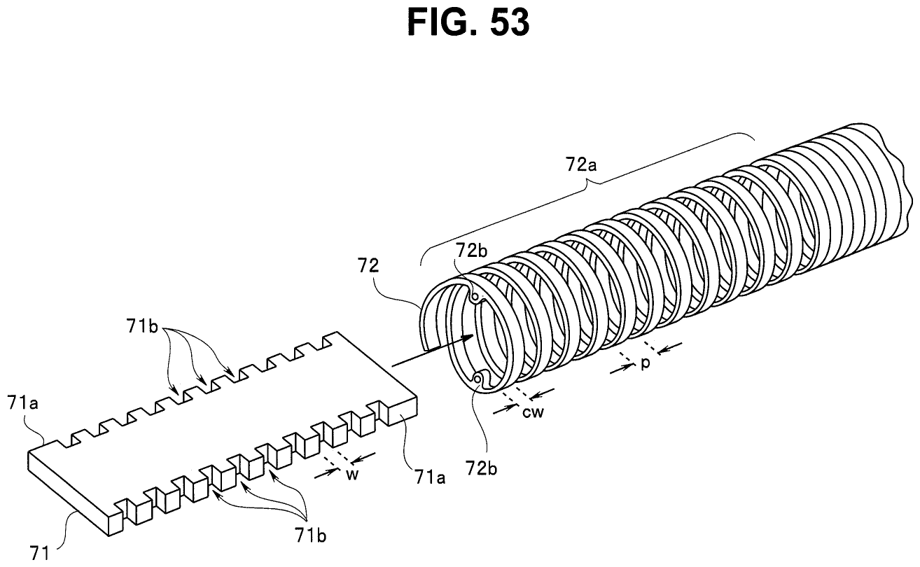

FIG. 52 is a configuration diagram of a small-diameter bending portion 7 according to the third disclosure example. FIG. 53 is an assembly diagram of a small-diameter bending portion 7 according to the third disclosure example. The bending portion 7 has a plate-shaped member 71 and a coil 72. The plate-shaped member 71 has a rectangular shape that is long in the longitudinal direction of the bending portion 7. The plate-shaped member 71 has both side surfaces 71a extending in the longitudinal direction. A plurality of recess portions 71b are formed on each side surface 71a in the longitudinal direction. Each recess portion 71b has a width w in the longitudinal direction.

The coil 72 has a coarsely wound coil portion 72a on the distal end side. The coarsely wound coil portion 72a is wound around the longitudinal axis at a winding pitch p. The coil 72 is made of an elongated thin plate member, and the thin plate member of the coil 72 has a width cw that allows the thin plate member to fit within the width w of the recess portion 71b of the plate-shaped member 71. Furthermore, the winding pitch p of the coil 72 has a pitch at which the coil 72 can engage with each recess portion 71b as shown in FIG. 52.

Two convex portions 72b are formed at the distal end portion of the coil 72, and the distal end portions of the two bending wires 26 are inserted into the holes formed in the convex portions 72b and fixed to the coil 72 by welding or the like.

According to such a configuration, the plate-shaped member 71 can be bent in the up-down direction by pulling or loosening the two bending wires 26, and the distal end side of the bending portion 7 easily bends by the coarsely wound coil portion 72a on the distal end side of the coil 72.

The present invention is not limited to the above-described embodiments, and various modifications, alterations, and the like can be made without changing the gist of the present invention.

What is claimed is:

1. An endoscope apparatus, comprising a bending portion bent by at least one bending wire,
the bending portion including a first region and a second region in a longitudinal direction of the bending portion,
the second region being a region provided on a proximal end side of the first region and configured to be bent following bending of the first region,
the at least one bending wire being held on a proximal end side of the second region,
the first region and the second region being integrally formed.

2. The endoscope apparatus according to claim 1, wherein
the first region includes a plurality of slits formed so as to allow the bending portion to be bent in a predetermined bending direction, and
the second region includes a tightly wound coil.

3. The endoscope apparatus according to claim 2, wherein the first region and the second region are integrally formed by being connected by welding.

4. The endoscope apparatus according to claim 2, wherein the plurality of slits in the first region and the tightly wound coil in the second region are formed by processing one pipe.

5. The endoscope apparatus according to claim 1, comprising
a flexible tube portion connected to a proximal end side of the bending portion,
wherein a Young's modulus of a first portion of the flexible tube portion is smaller than a Young's modulus of a second portion of the flexible tube portion, the first portion being adjacent to the bending portion, the second portion not being adjacent to the bending portion.

6. The endoscope apparatus according to claim 1, wherein
the bending portion includes a cylindrical pipe including the first region and the second region, and a multi-lumen tube inserted into the pipe, and
the at least one bending wire is inserted into a hole formed in the multi-lumen tube.

7. The endoscope apparatus according to claim 2, wherein the tightly wound coil includes a wire receiving portion configured to hold the at least one bending wire.

8. The endoscope apparatus according to claim 2, comprising a sheath member, the sheath member being a member covering the bending portion, the member including a first portion and a second portion, the first portion covering the plurality of slits, the second portion covering the tightly wound coil, the first portion having a Young's modulus smaller than a Young's modulus of the second portion.

9. The endoscope apparatus according to claim 2, wherein
the plurality of slits are formed in a pipe member, and
a second thin-walled portion of the tightly wound coil has a greater thickness than a first thin-walled portion of the pipe member.

10. The endoscope apparatus according to claim 2, wherein the bending portion includes another tightly wound coil on a distal end side of the first region.

11. The endoscope apparatus according to claim 1, wherein
the first region includes a coarsely wound coil and a plurality of pipes fixed inside the coarsely wound coil, and
the second region includes a tightly wound coil.

12. The endoscope apparatus according to claim 2, wherein the first region and the second region are formed by processing a multi-lumen tube.

13. The endoscope apparatus according to claim 2, wherein the first region in plurality and the second region in plurality are each provided so as to be arranged alternately.

14. The endoscope apparatus according to claim 4, wherein the plurality of slits are formed so as to increase bendability from the tightly wound coil toward a distal end of the plurality of slits.

15. The endoscope apparatus according to claim 14, wherein the plurality of slits include, in the longitudinal direction of the bending portion, parts in which pitches between two adjacent slits of the plurality of slits are different.

16. The endoscope apparatus according to claim 14, wherein the plurality of slits include, in the longitudinal direction of the bending portion, parts in which lengths of the plurality of slits in circumferential direction of the bending portion are different.

17. The endoscope apparatus according to claim 14, wherein the plurality of slits include, in the longitudinal direction of the bending portion, parts in which widths of the plurality of slits are different.

18. A bending member for endoscope, comprising a first region and a second region in a longitudinal direction of a bending portion of an endoscope,
the second region being a region provided on a proximal end side of the first region and configured to be bent following bending of the first region,
the first region and the second region being integrally formed.
